(19) United States
(12) Patent Application Publication
(10) Pub. No.: US 2005/0281740 A1
(43) Pub. Date: Dec. 22, 2005

(54) Imaging damaged lung tissue

(76) Inventors: Glen Gong, San Francisco, CA (US); Ronald Dieck, Palo Alto, CA (US)

Correspondence Address: WILSON SONSINI GOODRICH & ROSATI, 650 PAGE MILL ROAD, PALO ALTO, CA, 94304-1050, US

(21) Appl. No.: 11/008,777

(22) Filed: Dec. 8, 2004

Related U.S. Application Data
(60) Provisional application No. 60/580,444, filed on Jun. 16, 2004.
(60) Provisional application No. 60/586,932, filed on Jul. 8, 2004.
(60) Provisional application No. 60/586,950, filed on Jul. 8, 2004.

(51) Int. Cl. A61K 51/00

(52) U.S. Cl. 424/1.69

(57) ABSTRACT

The present invention relates to methods and compositions for targeting damaged lung tissue. Compositions provided feature a targeting moiety coupled to one or more other moieties, including, for example, a cross-linkable moiety, an imaging moiety, and/or one or more other targeting moieties. The methods and compositions of the invention find use, for example, in detecting and treating a pulmonary condition such as emphysema.

### RELATED APPLICATIONS

This application claims priority to provisional applications U.S. 60/580,444, entitled “Targeting Damaged Lung Tissue,” filed Jun. 16, 2004; U.S. 60/586,932, entitled “Targeting Damaged Lung Tissue Using Various Formulations,” filed Jul. 8, 2004; and U.S. 60/586,950, entitled “Lung Volume Reduction Using Glue Composition,” filed Jul. 8, 2004, each of which is incorporated herein in its entirety.

### BACKGROUND OF THE INVENTION

Pulmonary conditions affect millions of Americans and many more individuals worldwide. Chronic obstructive pulmonary disease (COPD), for example, including emphysema, asthma, bronchiectais and chronic bronchitis, is one of the most common chronic conditions and the fourth leading cause of death in the United States. While various environmental and genetic factors may contribute to COPD, cigarette smoking is the primary cause. Cigarette smoke can trigger inflammatory responses within the lungs, activating elastase, cathepsin G, and matrix metalloproteinases (MMPs). These enzymes are proteases that result in progressive destruction of the elastic tissue of the lungs, reducing the elasticity and lung recoil required for exhalation. Damaged alveolar walls can eventually rupture to form inelastic “blebs.” Emphysema, for example, is characterized by abnormal enlargement of alveolar airspaces distal to terminal bronchioles and destruction of airspace parenchyma resulting in such “blebs”.

Current diagnosis involves inference by a combination of factors, including history, pulmonary function, and radiology images (e.g., CT images), but the correlation of pulmonary function data with the extent of emphysema is poor. For example, radiograph is insensitive to mild emphysema and only about 40% of moderately severe emphysema and about 66% of severe emphysema show evidence of disease upon chest x-ray. Thus, there remains a need for improved methods for detecting and diagnosing pulmonary conditions, such as emphysema.

Current treatments are also wanting. Treatment of pulmonary conditions often involves control and management rather than a cure for the disease. With emphysema, for example, treatment can involve cessation of smoking, exercise programs, medications that help open constricted airways, anti-inflammatory medications, oxygen therapy, placement of one-way valves, and lung volume reduction surgery (LVRS). LVRS involves surgical removal of damaged, over-inflated lung tissue to free up space for the expansion of remaining non-damaged tissue. This technique requires, however, invasive procedures and benefits tend to decline over time. Further, treatments using one-way valves have not proved satisfactory. Thus, along with the need for better detection methods, there also remains a need for improved methods for treating pulmonary conditions, such as emphysema.

The present invention provides methods and compositions directed thereto. Other methods and compositions directed thereto are provided in U.S. nonprovisional applications entitled “Targeting Damaged Lung Tissue Using Compositions,” filed Dec. 8, 2004; “Targeting Damaged Lung Tissue,” filed Dec. 8, 2004; “Targeting Sites of Damaged Lung Tissue Using Composition,” filed Dec. 8, 2004; “Targeting Sites of Damaged Lung Tissue,” filed Dec. 8, 2004; “Imaging Damaged Lung Tissue Using Compositions,” filed Dec. 8, 2004; “Glue Compositions for Lung Volume Reduction,” filed Dec. 8, 2004; “Lung Volume Reduction Using Glue Compositions,” filed Dec. 8, 2004; “Glue Composition for Lung Volume Reduction,” filed Dec. 8, 2004; and “Lung Volume Reduction Using Glue Composition,” filed Dec. 8, 2004, each of which is herein incorporated in its entirely.

### BRIEF SUMMARY OF THE INVENTION

One aspect of the present invention relates to a method of imaging damaged lung tissue by administering to a subject a composition comprising an imaging moiety and a targeting moiety where the moieties are coupled and where the targeting moiety targets damaged lung tissue; and imaging the damaged lung tissue. In some embodiments, the lung tissue comprises epithelial lining fluid. In some embodiments, the composition does not comprise a polysaccharide or a carbohydrate moiety. In some embodiments, the composition does not comprise an antibody. In some embodiments, the composition does not comprise a mutant plasminogen activator-inhibitor type 1. In some embodiments, the composition does not comprise a lung membrane dipeptidase-binding molecule. In some embodiments, the targeting moiety does not target a lung membrane dipeptidase.

In some embodiments, the targeting moiety targets a damage-correlated moiety. In some embodiments, the damage-correlated moiety comprises a cell surface marker. In some embodiments, the damage-correlated moiety comprises an ECM component. In some embodiments, the targeting moiety targets elastase. In some embodiments, the targeting moiety targets neutrophil elastase. In some embodiments, the targeting moiety comprises a protease inhibitor moiety. For example, in some embodiments, the targeting moiety comprises an alpha-1 antitrypsin moiety, for example, a recombinant alpha-1 antitrypsin moiety. In some embodiments, the targeting moiety comprises an elafin moiety, for example, a recombinant elafin moiety. In some embodiments, the targeting moiety comprises a serpin moiety, for example, a recombinant serpin moiety, a secretory leukoprotease inhibitor (SLP1) moiety, and/or a recombinant secretory leukoprotease inhibitor (SLP1) moiety. In some embodiments, the targeting moiety targets at least one matrix metalloproteinase selected from MMP-1, MMP-2, MMP-3, MMP-4, MMP-5, MMP-6, MMP-7, MMP-8, and MMP-9. In some embodiments, the composition does not comprise a hyaluronic acid or a salt thereof. In some embodiments, the targeting moiety targets desmosine and/or isodesmosine. In some embodiments, the targeting moiety targets CD8 and/or CD4. In some embodiments, the targeting moiety targets a smoke-related moiety.

In some embodiments, the imaging moiety comprises a contrasting agent. In some embodiments, the contrasting agent is non-ionic. In some embodiments, the contrasting agent is ionic. In some embodiments, the imaging moiety comprises at least one metal compound selected from a tantalum compound and a barium compound. In some embodiments, the imaging moiety comprises iodine. In some embodiments, the imaging moiety comprises at least one organic iodo acid selected from an iodo carboxylic acid, a triiodophenol, an iodoform, and a tetraiodoethylene. In some embodiments, the imaging moiety comprises a non-radioactive moiety. In some embodiments, the imaging moiety comprises a proton-emitting moiety. In some embodiments, the imaging moiety comprises a radiopaque moiety and/or a radioactive moiety. In some embodiments, the imaging moiety comprises a magnetic moiety. In some embodiments, the imaging moiety comprises a radiopharmaceutical. In some embodiments, the imaging moiety comprises an In-111 moiety. In some embodiments, the imaging moiety comprises a Tc-99m moiety. In some embodiments, the imaging moiety comprises an Xe-133 moiety.

In some embodiments, the composition is less than 10 microns. In some embodiments, the composition is less than 5 microns. In some embodiments, the composition is less than 1 micron.

In some embodiments, the administering is carried out via inhalation, for example, the inhalation is carried out via the mouth. In some embodiments, the administering is carried out via trans-thoracic administration. In some embodiments, the administering is carried out via intravenous administration.

In some embodiments, the imaging of the damaged lung tissue is carried out via a radiological technique. In some embodiments, the radiological technique is at least one selected from an X-ray, a CT scan, a PET scan, a nuclear scan, a SPECT, and a scintigraphy.

In some embodiments, the composition further comprises a coupled cross-linkable moiety and/or another coupled targeting moiety. In some embodiments, the method further comprises cross-linking and/or sealing the damaged lung tissue. In some embodiments, the method further comprises administering a washing moiety.

Another aspect of the invention relates to a method of diagnosing a pulmonary condition by administering to a subject a composition comprising an imaging moiety and a targeting moiety where the moieties are coupled and where the targeting moiety targets damaged lung tissue; and imaging the damaged lung tissue. In some embodiments, the lung tissue comprises epithelial lining fluid. In some embodiments, the pulmonary condition is emphysema. In some embodiments, the pulmonary condition is COPD.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1a illustrates one embodiment of a method to reduce lung volume using a composition comprising a cross-linkable moiety coupled to a targeting moiety that targets damaged lung tissue; FIG. 1b illustrates one embodiment of a method to reduce lung volume using a composition comprising coupled targeting moieties that target different sites of damaged lung tissue.

FIG. 2 illustrates one embodiment of a method to image damaged lung tissue using a composition comprising an imaging moiety coupled to a targeting moiety that targets damaged lung tissue.

### DETAILED DESCRIPTION OF THE INVENTION

One aspect of the present invention provides a composition comprising a targeting moiety that targets damaged lung tissue, including lung fluids, such as, for example, epithelial lining fluid. A targeting moiety may preferentially or selectively target damaged lung tissue, for example, sites of diseased and/or non-normal lung tissue that may be affected, have been affected, or are likely to be affected by a pulmonary condition. In preferred embodiments, the targeting moiety recognizes and/or binds to a damage-correlated moiety that may occur in higher amounts in areas of the lung affected by a pulmonary condition compared with areas of the lung that are not affected or that are affected to a lesser extent. Targeting damaged lung tissue, and its various grammatical conjugations, as used herein includes targeting such damage-correlated moieties, e.g. any and all of the damage-correlated moieties disclosed herein and/or incorporated by reference. Affected areas of damaged lung tissue can also include lung fluids, such as, for example, epithelial lining fluid. Such damaged-correlated moieties are present in the lungs of the subject due to, e.g., disease progression, and need not be administered to the subject, e.g., prior to administration of the composition comprising the targeting moiety.

Targeting, including preferential and/or selective targeting, does not mean that the targeting moiety does not bind to any normal and/or non-damaged areas of the lung or to any other non-lung tissues. In some embodiments, targeting means, for example, being at least about 20-fold, at least about 30-fold, at least about 50-fold, at least about 75-fold, at least about 100-fold, at least about 150-fold, or at least about 200-fold selective for a corresponding damage-correlated moiety in terms of relative Kᵢ over other lung tissue components. In some embodiments, the targeting moiety has at least about a 50-fold selectivity, at least about a 100-fold selectivity, at least about a 200-fold selectivity, at least about a 300-fold selectivity, at least about a 400-fold selectivity, at least about a 500-fold selectivity, at least about a 600-fold selectivity, at least about a 700-fold selectivity, at least about an 800-fold selectivity, at least about a 1000-fold selectivity, or at least about a 1500-fold selectivity to a corresponding damage-correlated moiety. For example, in some preferred embodiments, the targeting moiety has a K₁ value against a damage-correlated moiety of less than about 200 nM, less than about 150 nM, less than about 100 nM, or less than about 75 nM. In some preferred embodiments, the targeting moiety has a Kᵢ value against a damage-correlated moiety of more than about 50 nM, more than about 25 nM, more than about 20 nM, more than about 15 nM, more than about 10 nM, more than about 5 nM, more than about 3 nM, or more than about 1 nM. In some preferred embodiments, the targeting moiety binds its target damage-correlated moiety with a K_{D} less than about 10⁻⁸ M, less than about 10⁻⁹ M, less than about 10⁻¹⁰ M, less than about 10⁻¹¹ M, less than about 10⁻¹² M, less than about 10⁻¹³ M, or less than about 10⁻¹⁴ M.

Binding in the context of a targeting moiety recognizing and/or binding to its target damage-correlated moiety can refer to both covalent and non-covalent binding, for example where a targeting moiety may bind, attach or otherwise couple to its target damage-correlated moiety by covalent and/or non-covalent binding. Binding may be either high affinity or low affinity, preferably high affinity. Examples of binding forces that may be useful in the present invention include, but are not limited to, covalent bonds, dipole interactions, electrostatic forces, hydrogen bonds, hydrophobic interactions, ionic bonds, and/or van der Waals forces.

“Damage-correlated moieties” include, for example, substances found at higher concentrations in lung tissue affected by a pulmonary condition than in areas of the lung that are not affected or that are affected to a lesser extent. As used herein, the terms “area,” “region” and “site” are used interchangeably when referring to regions, sites and/or areas of damaged lung tissue. For example, the damage-correlated moiety may be found attached, bound, coupled, complexed and/or otherwise associated with lung tissue affected by a pulmonary condition at higher concentrations than in areas of the lung that are not affected or that are affected to a lesser extent. Binding, attachment, coupling, complexing and/or association may involve covalent and/or non-covalent interactions, including, e.g., dipole interactions, electrostatic forces, hydrogen bonds, hydrophobic interactions, ionic bonds, and/or van der Waals forces.

In some embodiments, the damage-correlated moiety is bound, attached, coupled, complexed and/or otherwise associated with a cell surface of lung tissue affected by a pulmonary condition at higher concentrations than in areas of the lung that are not affected or that are affected to a lesser extent. In some embodiments, the damage-correlated moiety may be bound to a cell wall. In some embodiments, the damage-correlated moiety may be complexed with a moiety that is itself bound to a cell wall. In some embodiments, the damage-correlated moiety may comprise a cell surface marker, e.g., where the cell surface marker is associated with lung tissue affected by a pulmonary condition in that, e.g., where the cell surface marker is found at higher concentrations in areas of lung tissue affected by a pulmonary condition than in areas of the lung that are not affected or that are affected to a lesser extent.

In still some embodiments, the damage-correlated moiety may be found associated with the extra cellular matrix (ECM) at higher concentrations in areas of lung tissue affected by a pulmonary condition than in areas of the lung that are not affected or that are affected to a lesser extent. For example, a damage-correlated moiety may comprise an ECM component or may be associated with an EMC component of lung tissue affected by a pulmonary condition at higher concentrations than in areas of the lung that are not affected or that are affected to a lesser extent.

In some embodiments, a damage-correlated moiety comprises at least one moiety selected from a protein moiety, a glycoprotein moiety, a lipoprotein moiety, a lipid moiety, a phospholipid moiety, a carbohydrate moiety, a nucleic acid moiety, a modified nucleic acid moiety, and/or a small molecule moiety, including, e.g., a cell surface marker comprising a glycoprotein moiety and/or an ECM component comprising a protein moiety.

In some embodiments, damage-correlated moieties comprise proteases found at higher concentrations in lung tissue affected by a pulmonary condition than in areas of the lung that are not affected or that are affected to a lesser extent. For example, in some preferred embodiments, the targeting moiety targets elastase. The elastase may be bound to the cell wall and/or associated with the extracellular matrix of lung tissue affected by a pulmonary condition at higher concentrations than in areas of the lung that are not affected or that are affected to a lesser extent. For example, elastase causes progressive destruction of elastic fibers of lung tissues in some pulmonary conditions, e.g., emphysema, resulting in dilation and rupture of distended alveoli to form characteristic “blebs.” Suki et al., “On the Progressive Nature of Emphysema, Pulmonary Perspective”, American Journal of Respiratory and Critical Care Medicine, Vol. 168 pgs. 516-520 (2003); Janoff et al., Am. Rev. Respir. Dis., Vol. 132 pgs. 417-433 (1985); Senior and Kuhn, In Fishman (ed), Pulmonary Diseases and Disorders, 2d ed. N.Y., McGraw-Hill, p. 1209-1218 (1988). In some preferred embodiments, the targeting moiety targets neutrophil elastase and/or neutrophils. In some preferred embodiments, the targeting moiety targets pancreatic and/or macrophage elastase. In some preferred embodiments, the targeting moiety targets neutrophil proteinase 3 (Pr3). Pr3 is descried, for example, in Duranton et al., “Inhibition of proteinase 3 by alpha-1 antitrypsin in vitro predicts very fast inhibition in vivo”, Am J Respir Cell Mol. Biol., Vol. 29 No. 1 pgs 57-61 (2003).

For example, the targeting moiety may (or may not) comprise alpha-1 antitrypsin, elafin, thypin (see, e.g., International Publication No. WO 02/072769), and/or other serpin, e.g., PAI-1, PAI-2, SCCA-1, SCCA-2, secretory leukoprotease inhibitor SLP-1, HMCIS41 (see, e.g., U.S. Pat. No. 6,753,164), and/or other serpin-related proteins (e.g., as disclosed in U.S. Publication No. 2004/0126777); a recombinant form of any of these and/or a moiety of any of these that retains the ability to recognize and/or bind to its target. In some embodiments, the targeting moiety may (or may not) comprise mucous proteinase inhibitor (MPI) that shows high affinity for binding to elastase. Belotgey et al., “Effect of polynuclotides on the inhibition of neutrophil elastase by mucus proteinase inhibitor and alpha-1 proteinase inhibitor”, Biochemistry, Vol. 37 No. 46 pgs 16416-22 (1998). Other targeting moieties that can target elastase may also be used, such as inhibitors of elastase known in the art. See, e.g., Janoff et al., Am. Rev. Respir. Dis. Vol. 132 pgs 417-433 (1985); Zimmerman and Powers (1989), In Hornebeck (ed), Elastin and Elastases, vol II, Boca Raton, CRC Press, pgs 109-123; and Laurell and Eriksson Scand. J. Clin. Lab. Invest., Vol. 15 pgs 132-140 (1963). Other targeting moieties may or may not include protease inhibitors of the inter-alpha trypsin inhibitor (ITI) family. The ITI protein family can be built up from different combinations of the polypeptides HC1, HC2, HC3 and bikunin, as described, e.g., in Cuvelier et al., “Proteins of the inter-alpha trypsin inhibitor (ITI) family. A major role in the biology of the extracellular matrix”, Rev Mal Respir., Vol. 17 No. 2 pgs 437-46 (2000).

Alpha-1 antitrypsin useful for preparing a targeting moiety of the present invention may be obtained by any techniques known in the art and/or disclosed herein. For example, alpha-1 antitrypsin can be obtained by recombinant methods, as known in the art (e.g., recombinant alpha-1 antitrypsin from Novartis). Techniques for purifying alpha-1 antitrypsin, e.g., from biological natural and/or recombinant sources are also known in the art. See, e.g., International Publication No. WO 00/17227 and U.S. Pat. No. 4,656,254, which describes separating alpha-1 antitrypsin from plasma.

In some preferred embodiments, the targeting moiety targets desmosine and/or isodesmosine. Desmosine and/or isodesmosine are amino acids produced as a result of damage to lung tissues, particularly damage involving destruction of elastin. Fragmented elastin, for example, is metabolized to free desmosine or small peptides, which can be recovered in the urine of the subject. See, e.g., Starcher B. C., “Lung Elastin and Matrix”, Chest, Vol. 117 pgs. 229S-234S (2000). In animal models of emphysema, for example, desmosine urine recovery can serve as a measure of lung damage. There are several micromethods for measuring desmosine, including, for example, enzyme-linked immunosorbent assay (see, e.g., Osakabe T. et al. “Comparison of ELISA and HPLC for the determination of desmosine and isodesmosine in aortic tissue elastin”, J Clin Lab Anal Vol. 9 pgs 293-296 (1995)); isotope dilution (see, e.g., Stone P. J. et al. “Measurement of urinary desmosine by isotope dilution and high performance liquid chromatography”, Am Rev Respir Dis Vol. 144 pgs 284-290 (1991)); high performance liquid chromatography (see, e.g., Covault H. P. et al. “Liquid-chromatographic measurement of elastin”, Clin Chem Vol. 28 pgs 1465-1468 (1982)); and/or radioimmunoassay (see, e.g., Starcher B. “A role for neutrophil elastase in the progression of solar elastosis”, Connect Tissue Res Vol. 31 pgs 133-140 (1995)). Targeting desmosine and/or isodesmosine in the lungs can direct a targeting moiety to sites of lung damage, e.g., as desmosine and/or isodesmosine may be found at higher amounts in areas of the lung affected by a pulmonary condition compared with areas of the lung that are not affected or that are affected to a lesser extent.

In some preferred embodiments, the targeting moiety targets cathepsin, e.g., cathepsin G, which can be produced by inflammatory cells in the pathogenesis of COPD. In some embodiments, the targeting moiety targets other cysteine proteinases. In some embodiments the targeting moiety targets cathepsins L, S, and K. In some embodiments, the targeting moiety targets RGS2, which accumulates at sites of macrophage activation, e.g., in activated-macrophage-related disorders, including emphysema. See, e.g., EP 1378518. In some embodiments, the targeting moiety targets alveolar macrophages. In some embodiments, the targeting moiety targets eosinophils. In some embodiments, the targeting moiety targets tumor necrosis factor-α. In some embodiments, the targeting moiety targets kallikrenin.

In some preferred embodiments, the targeting moiety targets a collagenase. The presence of collagenase activity may be detected, for example, by released components, e.g., amino acids, known to occur in collagen, e.g., hydroxyproline and/or hydroxylysine. Such components may occur in higher amounts in areas of the lung affected by a pulmonary condition compared with areas of the lung that are not affected or that are affected to a lesser extent and may serve as damage-correlated moieties for compositions of the present invention.

Examples of collagenases include, e.g., one or more metalloproteinases. Metalloproteinases include e.g., MMP-1 (interstitial collagenase or collagenase-1), MMP-2 (gelatinase-A or 72 kD gelatinase), MMP-3 (transin, human fibroblast stromelysin, or stromelysin-1), MMP-4, MMP-5, MMP-6, MMP-7 (matrilysin), MMP-8 (collagenase-2 or neutrophil collagenase), MMP-9 (gelatinase B or 92 kD gelatinase), MMP-10 (stromelysin II), MMP-11 (stromelysin III), MMP-12 (macrophase metalloelastase), and/or MMP-13 (collagenase-3), and as well as metalloproteinase ADAM 22 (see, e.g., U.S. Publication No. 2003/0194797). Metalloproteinases (also referred to as metalloproteases in the art) have been described, e.g., U.S. Publication No. 2003/0199440; U.S. Publication No. 2004/0048302; U.S. Publication No. 2004/0043407; U.S. Publication No. 2004/019479; and International Publication No. WO 02/072751. For example, a targeting moiety comprising an ilomastat moiety may be used. See, e.g., International Publication No. WO 2004/052236.

In some embodiments, the composition does not comprise a polysaccharide or carbohydrate moiety, e.g., in some embodiments, the composition does not comprise hyaluronic acid or a salt thereof; and in some embodiments, the composition does not comprise dextran or glycosaminoglycan. In some embodiments, the composition does not comprise a polysaccharide or carbohydrate moiety that binds to elastic fibers. In some embodiments, the composition does not comprise an antibody. In some embodiments, the composition does not comprise a lung membrane dipeptidase-binding molecule, e.g., in some embodiments, the composition may not target lung membrane dipeptidase, and in some embodiments the composition may not comprise GFE-1 peptide. See, e.g., Ruoslahti et al., “Membrane dipeptidase is the receptor for a lung-targeting peptide identified by in vivo phage display”, J Biol Chem Vol. 274 No. 17 pgs 11593-8 (1999) and U.S. Pat. No. 6,784,153.

Also, in some preferred embodiments, the targeting moiety targets CD8 and/or CD4, CD8 lymphocytes and/or CD4 lymphocytes, and/or interleukin 8 (see, e.g., U.S. Publication No. 2003/0232048). In some embodiments, the targeting moiety targets mitogen-activated protein kinase (see, e.g., International Publication No. WO 03/064639). In some embodiments, the targeting moiety may (or may not) target CIRL-2 homologs (see, e.g., International Publication No. WO 2004/031235). In still some embodiments, the targeting moiety may (or may not) comprise an antibody and/or binding fragment thereof that targets a damaged-correlated moiety. For example, the targeting moiety may comprise a COPD-related human Ig derived protein, discussed e.g. in International Publication No. WO 02/072788 and/or U.S. Publication No. 2003/0017150, which can recognize and/or bind COPD related proteins found at higher amounts in areas of the lung affected by a pulmonary condition compared with areas of the lung that are not affected or that are affected to a lesser extent. In yet another example, the targeting moiety may comprise an antibody to secreted protein HCEJQ69 (see, e.g., U.S. Pat. No. 6,774,216).

Preferred targeting moieties of the present invention comprise biological moieties, such proteins or polypeptides, which recognize and/or bind damage-correlated moieties in the lung, and can include naturally-occurring inhibitors of damage-correlated moieties, such as alpha-1 antitrypsin and/or mutants thereof and/or fragments thereof as well as other protease inhibitor moieties. As well as alpha-1 antitrypsin, other naturally-occurring inhibitors of elastase may also be used as preferred targeting moieties of the present invention, including, e.g., monocyte elastase inhibitor and variants thereof (see, e.g., International Publication No. WO 96/10418; U.S. Pat. No. 5,827,672; U.S. Pat. No. 5,663,299); as well as tissue inhibitors of metalloproteinases (TIMPs), such as TIMP-1, TIMP-2, TIMP-3, and TIMP-4. In more preferred embodiments, the targeting moiety is modified such that it binds to its target damage-correlated moiety irreversibly, substantially irreversibly, or at least with a high binding constant, e.g., to resist dissociation for a desired period of time. Targeting moieties may be selected and/or developed to increase binding affinity for a target damage-correlated moiety. For example, alpha-1 antitrypsin may be mutated by random and/or directed synthesis, to engineer mutants with higher binding constants for its target elastase.

Other non-naturally occurring inhibitors of damaged-correlated moieties that may (or may not) be used as a targeting moiety of the present invention include inhibitors of neutrophil elastase (e.g., methyl ketone derivatives); inhibitors of macrophage metalloproteinase (e.g., RS113456 and inhibitors discussed in U.S. Publication No. 2003/0199440); Cathepsin G inhibitors (e.g., LEX-032 (Sparta)); various elastase inhibitors (e.g.ABT-491 (Abbot)); inhibiting compositions (e.g., as disclosed in U.S. Publication No. 2003/0199440 and International Publication No. WO 03/090682, including lipase inhibitors and phospholipase inhibitors); protease inhibitor compositions (e.g., as disclosed in International Publication No. WO 2004/045634); Erdosteine (Edmond Pharma), FK-706 (Fujisawa), GW-311616 (Glaxo-Wellcome), Midesteine (Medea); a mutant plasminogen activator-inhibitor type 1, which can inhibit neutrophil elastase (e.g., U.S. Publication No. 2003/0216321); an N-substituted azetidinone (e.g., EP 0529719); peptidyl carbamates (e.g., U.S. Pat. No. 5,008,245 and/or EP 0367415), SR-268794 (Sanoti), and/or SYN-1134 (Syn. Pharm.); other proteinase inhibitors (e.g., CMP-777 (Dupont)); and benzamide and sulfonamide substituted aminoguanidines and alkoxyguanidines (see, e.g., U.S. 2004/0106633 and EP 1070049); as well as ON-elastase inhibitors (e.g., NX-21909 (Gilead)); and several HNE inhibitors (e.g., CE-1037 (Cortech/United Ther), CE-2000 series (Cortech/Ono), EPI-HNE-4 (Dyax), EPI-HNE-1 (Protein Engineer), MDL-101146 (HMR), Ono-5046 (Ono), SPAAT (UAB Res. Found.), WIN-63759 (Sterling Winthrop), ZD-8321 (AstraZeneca), and/or ZD-0892 (AstraZeneca)). Targeting moieties may (or may not) also include inhibitors and/or antibodies of any damage-correlated moieties described herein, as well as inhibitors and/or antibodies of proteins described in International Publication No. WO 03/010327; as well as inhibitors and/or antibodies of eosinophil serine protease 1-like enzymes described in U.S. Publication No. 2003/0224430 and/or other serine proteases, e.g., described in International Publication No. WO 2004/053117; as well as inhibitor and/or antibodies of transmembrane serine proteases, e.g., as discussed in U.S. Pat. No. 6,734,006; as well as inhibitors and/or antibodies of esterase described in International Publication No. WO 04/020620. As used herein, “antibodies” includes binding fragments thereof.

In some preferred embodiments, the targeting moiety comprises a compound, such as a small molecule compound, that targets a damage-correlated moiety. Such compounds can be obtained, for example, via ligand screening methods, as known in the art, using a damaged-correlated moiety as the target. For example, a biological sample or a defined candidate moiety can be brought into contact with a damaged-correlated moiety, for example purified and/or recombinant elastase, or fragments thereof, as well as a damage-correlated moiety isolated and/or purified from epithelial lining fluid. The candidate moiety may be labeled with a detectable label, such as a fluorescent, radioactive, and/or an enzymatic tag and allowed to contact the damage-correlated moiety that may be immobilized, e.g., under conditions that permit binding, e.g., selective and/or preferential binging. After removing unbound moieties, bound moiety can be detected using appropriate methods as known in the art.

Candidate moieties that can be assayed for targeting a damage-correlated moiety for use in the present invention are not limited. For example, such candidate moieties can be obtained from a wide variety of sources including libraries of synthetic, semi-synthetic and/or natural substances. Random and/or directed synthesis can be used, for example, to generate a wide variety of organic compounds and biomolecules, including randomized oligonucleotides and oligopeptides. With respect to natural compounds, libraries form bacterial, fungal, plant and animal extracts are available and/or can be readily produced. Further, natural, semi-synthetically, and/or synthetically produced libraries can be modified through conventional chemical, physical, recombinant, and/or biochemical techniques to produce combinatorial libraries. Also, known pharmaceutical or pharmacological agents may be modified by directed or random chemical modifications, including, for example, acylation, amidification, alkylation, and/or esterification to produce structural analogs.

Candidate moieties may include natural, synthetic and/or semi-synthetic organic compounds, macromolecules of biological origin, such as polypeptides, peptides, polysaccharides, glycoproteins, lipoproteins, fatty acids, and/or fragments thereof; and/or drugs or small molecules, such as molecules generated through combinatorial chemistry approaches. Further, when the candidate moiety comprises a peptide or polypeptide, the candidate moiety may be expressed by a phage clone belonging to a phage-based random peptide library (see, e.g., Parmley and Smith, Gene Vol. 73 pgs 305-318 (1988); Oldenburg et al., Proc. Natl. Acad. Sci. USA Vol. 89 pgs 5393-5397(1992); Valadon et al., J. Mol. Biol., Vol. 261 pgs 11-22 (1996); Westerink, Proc. Natl. Acad. Sci USA., Vol. 92 pgs 4021-4025 (1995); and Felici et al., J. Mol. Biol., Vol. 222 pgs 301-310) (1991); and/or the candidate moiety may be expressed from a cDNA cloned in a vector for performing a two-hybrid screening assay (U.S. Pat. Nos. 5,667,973 and 5,283,173; Harper et al., Cell, Vol. 75 pgs 805-816 (1993); Cho et al., Proc. Natl. Acad. Sci. USA, Vol. 95(7) pgs 3752-3757 (1998); and Fromont-Racine et al., Nature Genetics, Vol. 16(3) pgs 277-282 (1997).

Further, it is to be understood that the targeting moiety may target one of more types of damage-correlated moieties, including any combination of the damaged-correlated moieties disclosed herein, for example, one or more proteases and/or one or more smoke-related moieties as described below.

“Damage-correlated moieties” can also include a smoke-related moiety. For example, the targeting moiety may recognize and/or bind to cigarette smoke particles, tar, tobacco, and/or other smoke-related residues, such as Cadmium, that may be found in higher amounts in areas of the lung affected by a pulmonary condition compared with areas of the lung that are not affected or that are affected to a lesser extent.

Still other damage-correlated moieties can also include modified polypeptides, where the modification occurs at higher amounts in areas of the lung affected by a pulmonary condition compared with areas of the lung that are not affected or that are affected to a lesser extent. For example, members of the G-protein coupled receptor (GPCR) family, e.g., RAI-3 are modified, e.g., phosphorylated, and/or associated with tyrosine phosphorylated activation complexes following exposure to cigarette smoke. See, e.g., International Publication No. WO 04/001060 and/or U.S. Publication No. 2004/0121362. In some embodiments of the present invention, a targeting moiety may be used that targets such modified proteins and/or protein complexes. Such targeting moieties may (or may not) include modulators of RAI-3, as described in U.S. Publication No. 2004/0121362. In still some embodiments, a targeting moiety may (or may not) be used that targets polypeptides associated with the NF-KB pathway that are found in lung tissue, e.g., as described in U.S. Publication No. 2004/0086896.

Other damage-correlated moieties can include moieties that inhibit the production of elastic and/or connective tissue proteins. Such moieties may include, e.g., moieties that inhibit fibroblast proliferation and/or that inhibit procollagen production and/or that inhibit proteoglycan synthesis, preferably moieties that inhibit synthesis of the major matrix-associated proteoglycans, such as versican, decorin, and/or large heparan sulfate proteoglycans. “Inhibiting” and its various grammatical conjugations can mean reducing a biological process, e.g., reducing synthesis of a connective tissue component, by an amount compared with the occurrence of the process in the absence (or in the presence of lower levels) of the damage-correlated moiety. In some embodiments, the amount may be reduced by at least about 10%, at least about 20%, at least about 30%, at least about 40%, or at least about 50%. In some embodiments, the amount may be reduced by less than about 60%, less than about 70%, less than about 80%, less than about 90%, or less than about 95%. “Inhibiting” and its various grammatical conjugations need not mean completely inhibiting a biological process, e.g., it need not mean inhibiting synthesis of a connective tissue component to negligible and/or non-detectable levels. Damage-correlated moieties that can inhibit proteoglycan synthesis include, for example, Cadmium. See, e.g., Chambers et al., “Cadmium inhibits proteoglycan and procollagen production by cultures human lung fibroblasts,” Am. J. Respir. Cell Mol. Biol., Vol. 19 No. 3 pgs 498-506 (1998). Other damage-correlated moieties may include lead, aldehydes and/or silicates. Fujiwara, “Cell biological study on abnormal proteoglycan synthesis in vascular cell exposed to heavy metals,” Journal of Health Science, Vol. 50 No. 3 pgs 197-204 (2004). Still other damage-correlated moieties can include moieties that impair the repair of elastic and/or connective tissues of the lungs.

In some aspects of the present invention, a composition comprising a targeting moiety also comprises a cross-linkable moiety coupled thereto. The cross-linkable moiety can be any moiety that facilitates linkage between more than one cross-linkable moieties, preferably between cross-linkable moieties coupled to targeting moieties binding to damage-correlated moieties at different sites of damaged lung tissue. Cross-linkable moieties can include, for example, a hydroxyl group, carboxyl group, ester group, cyano group, thiol group (including e.g., a cysteine group), carbonyl group, aldehyde group, ketone group, primary amine group, and/or secondary amine group, as well as a lysine group.

In some embodiments, the cross-linkable moiety comprises any other amine groups, a sulfide group, a carbonyl group (e.g., α-halocarbonyl group and/or α,β-unsaturated carbonyl group), a cyanate group (e.g., isothiocyanate group), a carboxylate group (e.g., an acetate group such as α-haloacetate group), a hydrazine group, and/or a biotin group,. See, e.g., US Publication No. 2002/0071843.

In some embodiments, the cross-linkable moiety can comprise fibrinogen and/or fibrin. Fibrinogen can be converted to fibrin, which is polymerized in a cross-linking reaction. In some embodiments, the cross-linkable moiety can comprise other protein and/or proteinaceous materials, e.g., proteinaceous materials comprising albumin (bovine or human), collagen, PEI, oleic acid, chitin and/or chitosan, as well as any of those described in U.S. Pat. No. 5,385,606, U.S. Pat. No. 5,583,114, U.S. Pat. No. 6,310,036, U.S. Pat. No. 6,329,337, and/or U.S. Pat. No. 6,372,229. In some embodiments, more than one type of cross-linkable moiety may be coupled to a given targeting moiety or may be coupled to a number of targeting moieties used in combination, e.g., in one administration or in a number of successive administrations. Those of skill in the art will recognize other suitable cross-linkable moieties that may be used in the practice of the instant invention including, for example, any biocompatible cross-linkable moiety that can form a biocompatible cross-linked product.

The targeting moiety may be coupled to the cross-linkable moiety by any techniques and/or approaches known in the art, described herein, and/or as can be developed by those of skill in the art. For example, coupling methods include, but are not limited to the use of bifunctional linkers, amide formation, imine formation, carbodiimide condensation, disulfide bond formation, and/or use of a specific binding pair e.g., using a biotin-avidin interaction. These and other methods known in the art may be found, e.g., in Hermanson, “Bioconjugate Techniques,” Academic Press: New York, 1996; and S. S. Wong, “Chemistry of Protein Conjugation and Cross-linking,” CRC Press, 1993.

In preferred embodiments, the cross-linkable moiety is coupled to the targeting moiety in such a way so as not to interfere with the ability of the targeting moiety to target damaged lung tissue. For example the cross-linkable moiety can be attached to an alpha-1 antitrypsin moiety at one or more sites that do not modify the conformation or folding of the alpha-1 antitrypsin, or do not modify the conformation or folding of regions of alpha-1 antitrypsin necessary and/or involved in the recognition and/or binding to its damage-correlated moiety, e.g. elastase. For example, without being limited to a given hypothesis or mode of action, the active inhibitory site of alpha-1 antitrypsin is found around Ser358 of the polypeptide, e.g., forming a pseudo-irreversible equimolar complex with neutrophil elastase. See, e.g., Sifers et al., “Genetic Control of Human Alpha-1 Antitrypsin”, Mol. Biol. Med., Vol. 6 pgs 127-135 (1989). In some preferred embodiments, a cross-linkable moiety can be attached to an alpha-1 antitrysin moiety at a site other than around its Ser358 inhibitory site. Similarly, in some embodiments, without being limited to a given hypothesis or mode of action, a cross-linkable moiety can be attached to a serpin moiety at a site other than certain regions known to be involved in attaching to a target protease, which include, for example, the hinge, breach, shutter, and gate regions of serpins. Irving et al., Genome Res Vol. 10 pgs 1845-64 (2000). Some serpins, for example, contain a reactive center loop (RCL) involved in inhibition where a stable complex can be formed between the protease and a cleaved form of the serpin. Attachment to a site other than the RCL region of a serpin moiety is preferred in some embodiments. Similarly, in some embodiments, without being limited to a given hypothesis or mode of action, a cross-linkable moiety can be attached to a monocyte elastase inhibitor moiety at a site other than a cysteine residue of the inhibitor involved in interacting with its target elastase and/or proteinase 3 and/or cathepsin G. See, e.g., International Publication WO 96/10418; and U.S. Pat. No. 5,827,672.

In some embodiments, the cross-linkable moiety may be chemically bound to the targeting moiety, e.g., a carboxyl group covalently attached to one or more sites of alpha-1 antitrypsin. In some embodiments, the cross-linkable moiety may be chemically bound to a moiety that is itself chemically bound to the targeting moiety, indirectly coupling the cross-linkable and targeting moieties.

In preferred embodiments, the size of the composition comprising a targeting moiety coupled to a cross-linkable moiety is not so large as to prevent access of the composition to damage-correlated moieties, such as damage-correlated moieties within enlarged alveoli distal to a terminal bronchiole. For example, the size of the composition comprising a targeting coupled to a cross-linkable moiety is preferably less than about 10 microns, less than about 8 microns, less than about 5 microns, less than about 3 microns, less than about 2 microns, or less than about 1 micron. “Enlarged alveolus” as used herein refers to an alveolus that is larger than the average alveolus that is not affected by a pulmonary condition, or that is affected to a lesser extent. For example, an enlarged alveolus may be at least about 5%, at least about 10%, at least about 20%, at least about 50%, at least about 100%, or at least about 150% the size of an average alveolus.

Further, it is to be understood that a composition comprising a targeting moiety coupled to a cross-linkable moiety may further comprise a coupled or not coupled imaging moiety, e.g. depending on the intended use of the composition.

Another aspect of the present invention relates to a composition comprising a first targeting moiety and a second targeting moiety wherein said targeting moieties are coupled and wherein said targeting moieties can target different sites of damaged lung tissue. In preferred embodiments, the different sites comprise different sites within an enlarged air space, e.g., within alveolar walls of an over-inflated alveolus distal to a terminal bronchiole, as characteristic of some pulmonary conditions, including emphysema. For example, the first targeting moiety can target a first damage-correlated moiety while the second targeting moiety can target a second damage-correlated moiety, where the first and second damage-correlated moieties occur at different sites. The first and second targeting moieties may be the same or different, and the first and second damage-correlated moieties may be the same or different.

Further, it is to be understood that any plural number of targeting moieties may be used, i.e., the present invention also contemplates a composition comprising any plural number of coupled targeting moieties, that may each be the same or different, or some may be the same while others are different. For example, in a composition comprising three coupled targeting moieties, the first targeting moiety may be coupled to the second targeting moiety, which is coupled to a third targeting moiety. The first and third moieties may or may not be directly coupled to each other. In some embodiments, the three targeting moieties may be coupled to a moiety without being directly coupled to each other. The three moieties may all be the same or different, or two may be the same with the third is different. Each targeting moiety may target the same type of damage-correlated moiety, each may target a different type of damage-correlated moiety, or two may target the same type of damage-correlated moiety while the third targeting moiety targets a different type of damage-correlated moiety. The damage-correlated moieties targeted by the targeting moieties can occur at two or more different sites of damaged lung tissue, preferably, e.g., at different sites within an enlarged air space, e.g., within alveolar walls of an over-inflated alveolus distal to a terminal bronchiole.

The targeting moieties may be coupled by any techniques and/or approaches known in the art, described herein, and/or as can be developed by those of skill in the art. In some embodiments, coupling may involve covalent bonds, dipole interactions, electrostatic forces, hydrogen bonds, hydrophobic interactions, ionic bonds, van der Waals forces, and/or other bonds that can couple targeting moieties. For example, in some embodiments, targeting moieties are coupled via a coupling moiety, e.g., a chemical linker. Any chemical linker may be used, including, e.g., an aliphatic group covalently linking the targeting moieties. For example, a chemical linker useful in this invention may comprise two (or more) functional groups, where each of the functional groups can be chemically bonded to a targeting moiety, serving to couple the targeting moieties. Examples of functional groups include, e.g., a hydroxyl group, a carboxyl group, an ester group, a cyano group, a thiol group, a cysteine group, a carbonyl group, an aldehyde group, a ketone group, and/or an amine group, as well as a lysine group. Other functional groups include a cyanate group (e.g., isothiocyanate) and/or a carboxylate group (e.g., an acetate group such as α-haloacetate).

Other coupling techniques may also be used. For example, dimers and/or multimers of targeting moieties may be prepared using cross-linking techniques so that the targeting moieties are pre-cross-linked, e.g., forming one or more cross-links between cysteine residues of peptide and/or polypeptide targeting moieties. Linker length optimization techniques may also be used (see, e.g., U.S. Pat. No. 5,478,925), for use in the present invention.

In some embodiments, targeting moieties are coupled as a fusion polypeptide. For example, where the targeting moieties are peptides and/or polypeptides, two or more targeting moieties may be joined by a polypeptide linker as the coupling moiety, to form a fusion polypeptide or fusion protein. A fusion protein may be generated in various ways, including, e.g., chemical coupling and co-translation. In some preferred embodiments, targeting moieties are recombinantly expressed as a fusion product from a recombinant nucleic acid molecule, where the targeting moieties are linked, e.g., by one or more intervening amino acids, according to techniques known in the art. See, e.g., Francis, “Focus on Growth Factors”, Vol. 3 pgs 4-10 (Mediscript, London) (1992). Fusion proteins may also be made using other techniques known in the art, e.g., techniques used to create adzymes, which comprise an address binding site conjugated to a catalytic domain (e.g., as described in U.S. Publication No. 2004/0081648 and in U.S. Publication No. 2004/0081648); and/or by covalent linking (e.g., via disulfide bonds) between at least one amino acid of each coupled targeting moiety (e.g., as described in U.S. Publication No. 2004/0087778).

In some embodiments, the targeting moieties are coupled via a protein, e.g., via an antibody and/or a binding fragment thereof. In some embodiments, liposomes may be prepared that comprise a plural number of targeting moieties.

In some preferred embodiments, the targeting moieties are coupled in such a way so as not to interfere with the ability of the targeting moiety to target damaged lung tissue. For example two (or more) alpha-1 antitrypsin moieties can be coupled to each other at sites that do not modify the conformation or folding of the alpha-1 antitrypsin moieties, or do not modify the conformation or folding of regions of the alpha-1 antitrypsin moieties necessary and/or involved in the recognition and/or binding to its damage-correlated moiety, e.g. elastase. For example, without being limited to a given hypothesis or mode of action, the active inhibitory site of alpha-1 antitrypsin is found around Ser358 of the polypeptide, e.g., forming a pseudo-irreversible equimolar complex with neutrophil elastase. See, e.g., Sifers et al., “Genetic Control of Human Alpha-1 Antitrypsin”, Mol. Biol. Med., Vol. 6 pgs. 127-135 (1989). In some preferred embodiments, alpha-1 antitrysin moieties may be coupled to each other or other targeting moieties at sites other than around their Ser358 inhibitory sites. Similarly, in some embodiments, without being limited to a given hypothesis or mode of action, serpin moieties may be coupled to each other or other targeting moieties at sites other than certain regions known to be involved in attaching to target protease, which include, for example, the hinge, breach, shutter, and gate regions of serpins. Irving et al., Genome Res Vol. 10 pgs 1845-64 (2000). Some serpins, for example, contain a reactive center loop (RCL) involved in inhibition where a stable complex can be formed between the protease and a cleaved form of the serpin. Attachment via sites other than the RCL regions of serpin moieties is preferred in some embodiments. Similarly, in some embodiments, without being limited to a given hypothesis or mode of action, monocyte elastase inhibitor moieties can be coupled to each other or other targeting moieties at a site other than a cysteine residue of the inhibitor involved in interacting with its target elastase and/or proteinase 3 and/or cathepsin G. See, e.g., International Publication WO 96/10418 and U.S. Pat. No. 5,827,672.

In preferred embodiments, the size of the composition comprising two (or more) coupled targeting moieties is not so large as to prevent access of the composition to damage-correlated moieties, such as damage-correlated moieties within enlarged air spaces distal to a terminal bronchiole. For example, the size of the composition comprising two (or more) targeting moieties is preferably less than about 10 microns, less than about 8 microns, less than about 5 microns, less than about 3 microns, less than about 2 microns, or less than about 1 micron.

Coupling of the targeting moieties can keep the targeting moieties in close or relatively close physical proximity. For example, in some preferred embodiments a chemical linker may be used that comprises an aliphatic group of at least about 2 carbon atoms, at least about 5 carbon atoms, at least about 10 carbon atoms, or at least about 12 carbon atoms. In some preferred embodiments, a chemical linker that comprises an aliphatic group of less than about 30 carbon atoms, less than about 20 carbon atoms, or less than about 15 carbon atoms can be used. In some preferred embodiments, a polypeptide linker can be used that comprises at least about one amino acid, at least about 3 amino acids, or at least about 5 amino acids. In some preferred embodiments, a polypeptide linker that comprises less than about 12 amino acids, less than about 10 amino acids, or les than about 5 amino acids can be used.

Further, it is to be understood that a composition comprising two (or more) coupled targeting moieties may further comprise a coupled or not coupled cross-linkable moiety and/or a coupled or not coupled imaging moiety, e.g., depending on the intended use of the composition.

In other aspects of the present invention, the composition comprising a targeting moiety also comprises an imaging moiety coupled thereto. The imaging moiety can be any moiety that facilitates detection, either directly or indirectly, preferably by a non-invasive and/or in vivo visualization technique. For example, an imaging moiety may be detectable by any known imaging techniques, including, for example, a radiological technique. Imaging moieties can include, for example, a contrasting agent, e.g., where the contrasting agent is ionic or non-ionic. In some embodiments, for instance, the imaging moiety comprises a tantalum compound and/or a barium compound, e.g., barium sulfate. In some embodiments, the imaging moiety comprises iodine, such as radioactive iodine. In some embodiments, for instance, the imaging moiety comprises an organic iodo acid, such as iodo carboxylic acid, triiodophenol, iodoform, and/or tetraiodoethylene. In some embodiments, the imaging moiety comprises a non-radioactive imaging moiety, e.g., a non-radioactive isotope. For example, Gd can be used as a non-radioactive imaging moiety in certain embodiments.

Other examples of imaging moieties include moieties which emit or may be caused to emit detectable radiation (e.g., fluorescence excitation, radioactive decay, spin resonance excitation, etc.), moieties which affect local electromagnetic fields (e.g., magnetic, ferromagnetic, ferromagnetic, paramagnetic, and/or superparamagnetic species), moieties which absorb or scatter radiation energy (e.g., chromophores and/or fluorophores), quantum dots, heavy elements and/or compounds thereof. See, e.g., imaging moieties described in U.S. Publication No. 2004/0009122. Other examples of imaging moieties include a proton-emitting moiety, a radiopaque moiety, and/or a radioactive moiety, such as a radionuclide like Tc-99m and/or Xe-13. Such moieties can be used as a radiopharmaceutical. In still other embodiments, a composition of the present invention may comprise one or more different types of imaging moieties, including any combination of the imaging moieties disclosed herein.

Further examples of radioactive imaging moieties include gamma emitters, e.g., the gamma emitters In-111, I-125 and I-131, Rhenium-186 and 188, and Br-77 (see. e.g., Thakur, M. L. et al., Throm Res. Vol. 9 pg. 345 (1976); Powers et al., Neurology Vol. 32 pg. 938 (1982); and U.S. Pat. No. 5,011,686); positron emitters, such as Cu-64, C-11, and O-15, as well as Co-57, Cu-67, Ga-67, Ga-68, Ru-97, Tc-99m, In-113m, Hg-197, Au-198, and Pb-203. Other radioactive imaging moieties can include, for example tritium, C-14 and/or thallium, as well as Rh-105, I-123, Nd-147, Pm-151, Sm-153, Gd-159, Tb-161, Er-171 and/or Tl-201.

The use of Technitium-99m (Tc-99m) is preferable and has been described in other applications, for example, see U.S. Pat. No. 4,418,052 and U.S. Pat. No. 5,024,829. Tc-99m is a gamma emitter with single photon energy of 140 keV and a half-life of about 6 hours, and can readily be obtained from a Mo-99/Tc-99 generator.

In some embodiments, compositions comprising a radioactive imaging moiety can be prepared by coupling a targeting moiety with radioisotopes suitable for detection. Coupling may occur via a chelating agent such as diethylenetriaminepentaacetic acid (DTPA), 4,7,10-tetraazacyclododecane-N, N′, N″, N′″-tetraacetic acid (DOTA) and/or metallothionein, any of which can be covalently attached to the targeting moiety. In some embodiments, an aqueous mixture of technetium-99m, a reducing agent, and a water-soluble ligand can be prepared and then allowed to react with a targeting moiety of the present invention. Such methods are known in the art, see e.g., International Publication No. WO 99/64446. In some embodiments, compositions comprising radioactive iodine, can be prepared using an exchange reaction. For example, exchange of hot iodine for cold iodine is well known in the art. Alternatively, a radio-iodine labeled compound can be prepared from the corresponding bromo compound via a tributylstannyl intermediate.

Magnetic imaging moieties include paramagnetic contrasting agents, e.g., gadolinium diethylenetriaminepentaacetic acid, e.g., used with magnetic resonance imaging (MRI) (see, e.g., De Roos, A. et al., Int. J. Card. Imaging Vol. 7 pg. 133 (1991)). Some preferred embodiments use as the imaging moiety paramagnetic atoms that are divalent or trivalent ions of elements with an atomic number 21, 22, 23, 24, 25, 26, 27, 28, 29, 42, 44, 58, 59, 60, 61, 62, 63, 64, 65, 66, 67, 68, 69, or 70. Suitable ions include, but are not limited to, chromium(III), manganese(II), iron(II), iron(III), cobalt(II), nickel(II), copper(II), praseodymium(III), neodymium(III), samarium(III) and ytterbium(III), as well as gadolinium(III), terbiurn(III), dysoprosium(III), holmium(III), and erbium(III). Some preferred embodiments use atoms with strong magnetic moments, e.g., gadolinium(III).

In some embodiments, compositions comprising magnetic imaging moieties may be prepared by coupling a targeting moiety with a paramagnetic atom. For example, the metal oxide or a metal salt, such as a nitrate, chloride or sulfate salt, of a suitable paramagnetic atom can be dissolved or suspended in a water/alcohol medium, such as methyl, ethyl, and/or isopropyl alcohol. The mixture can be added to a solution of an equimolar amount of the targeting moiety in a similar water/alcohol medium and stirred. The mixture may be heated moderately until the reaction is complete or nearly complete. Insoluble compositions formed may be obtained by filtering, while soluble compositions may be obtained by evaporating the solvent. If acid groups on the chelating moieties remain in the composition of the present invention, inorganic bases (e.g., hydroxides, carbonates and/or bicarbonates of sodium, potassium and/or lithium), organic bases, and/or basic amino acids may be used to neutralize acidic groups, e.g., to facilitate isolation or purification of the composition.

In preferred embodiments, the imaging moiety is coupled to the targeting moiety in such a way so as not to interfere with the ability of the targeting moiety to target damaged lung tissue. For example the imaging moiety can be attached to an alpha-1 antitrypsin moiety at one or more sites that do not modify the conformation or folding of the alpha-1 antitrypsin moiety, or do not modify the conformation or folding of regions of the alpha-1 antitrypsin moiety necessary and/or involved in the recognition and/or binding to its damage-correlated moiety, e.g. elastase. For example, without being limited to a given hypothesis or mode of action, the active inhibitory site of alpha-1 antitrypsin is found around Ser358 of the polypeptide, and can form a pseudo-irreversible equimolar complex with neutrophil elastase. See, e.g., Sifers et al., “Genetic Control of Human Alpha-1 Antitrypsin”, Mol. Biol. Med., Vol. 6 pgs. 127-135 (1989). In some preferred embodiments, an imaging moiety can be attached to an alpha-1 antitrysin moiety at a site other than around its Ser358 inhibitory site. Similarly, in some embodiments, without being limited to a given hypothesis or mode of action, an imaging moiety can be attached to a serpin moiety at a site other than certain regions known to be involved in attaching to a target protease, which include, for example, the hinge, breach, shutter, and gate regions of serpins. Irving et al., Genome Res Vol. 10 pgs 1845-64 (2000). Some serpins, for example, contain a reactive center loop (RCL) involved in inhibition where a stable complex can be formed between the protease and a cleaved form of the serpin. Attachment to a site other than the RCL region of a serpin moiety is preferred in some embodiments. Similarly, in some embodiments, without being limited to a given hypothesis or mode of action, an imaging moiety can be attached to a monocyte elastase inhibitor moiety at a site other than a cysteine residue of the inhibitor involved in interacting with its target elastase and/or proteinase 3 and/or cathepsin G. See, e.g., International Publication WO 96/10418; U.S. Pat. No. 5,827,672.

In some embodiments, the imaging moiety may be chemically bound to the targeting moiety, e.g., an iodine moiety covalently attached to one or more sites of alpha-1 antrypsin. In some embodiments, the imaging moiety may be chemically bound to a moiety that is itself chemically bound to the targeting moiety, indirectly linking the imaging and targeting moieties.

In preferred embodiments, the size of the composition comprising a targeting moiety coupled to an imaging moiety is not so large as to prevent access of the composition to damage-correlated moieties, such as damage-correlated moieties within enlarged air spaces distal to a terminal bronchiole. For example, the size of the composition comprising a targeting and an imaging moiety is preferably less than about 10 microns, less than about 8 microns, less than about 5 microns, less than about 3 microns, less than about 2 microns, or less than about 1 micron.

In still other aspects of the present invention, the composition comprising a targeting moiety and an imaging moiety coupled thereto also comprises a coupled cross-linkable moiety and/or another coupled targeting moiety, where the size of the composition is preferably less than about 10 microns, less than about 8 microns, less than about 5 microns, less than about 3 microns, less than about 2 microns, or less than about 1 micron, and at least not so large as to prevent access of the composition to damage-correlated moieties, such as damage-correlated moieties within enlarged air spaces distal to a terminal bronchiole. In preferred embodiments, the cross-linkable moiety, other targeting moiety and/or imaging moiety are each coupled to the targeting moiety, either directly or indirectly, in such a way so as not to interfere with the ability of the targeting moiety to target damaged lung tissue. In some embodiments, the cross-linkable moiety and the imaging moiety may each be chemically bound to the targeting moiety. In some embodiments, the cross-linkable moiety and the imaging moiety may each be chemically bound to a moiety that is itself chemically bound to a targeting moiety, indirectly linking the cross-linkable, imaging, and targeting moieties. In still other embodiments, the cross-linkable moiety may be chemically bound to the targeting moiety, while the imaging moiety is chemically bound to a moiety that is itself chemically bound to the targeting moiety, or vice versa. In preferred embodiments comprising more than one coupled targeting moieties, one or more imaging moieties may be coupled directly or indirectly to one or more of the targeting moieties. Methods of Detecting and/or Treating Pulmonary Conditions

The present invention provides methods of detecting and/or treating pulmonary conditions using compositions that target damaged lung tissue. The term “pulmonary condition” as used herein refers to a non-normal condition of the lungs and/or lung tissue, for example, where there is damaged lung tissue. The term includes conditions characterized by a higher amount of one or more damage-correlated moieties in areas of the lung affected by the pulmonary condition compared with areas of the lung that are not affected or that are affected to a lesser extent. Examples of such pulmonary conditions include COPD, which includes emphysema (including both heterogeneous emphysema and homogenous emphysema, preferably heterogeneous emphysema), asthma, bronchiectais, and chronic bronchitis. Pulmonary conditions can also include other chronic pulmonary disorders, sarcoidosis, pulmonary fibrosis, pneumothorax, fistulae, bronchopleural fistulae, cystic fibrosis, inflammatory states, and/or other respiratory disorders. Pulmonary conditions can also include smoking-related and/or age-related changes to the lung, as well as lung damage caused by a traumatic event, infectious agents (e.g., bacterial, viral, fungal, tuberculin and/or viral agents), exposure to toxins (e.g., chemotherapeutic agents, environmental pollutants, exhaust fumes, and/or insecticides), and/or genetic factors (e.g., alpha-1 antitrypsin deficiency and other types of genetic disorders which involve elastic and/or connective tissues degradation and/or impaired synthesis of elastic and/or connective tissues and/or impaired repair of elastic and/or connective tissues of the lungs).

One aspect of the present invention provides a method of reducing lung volume by administering to a subject in need thereof a composition comprising a cross-linkable moiety coupled to a targeting moiety that targets damaged lung tissue, and cross-linking the damaged lung tissue. In some preferred embodiments, the method can be performed without prior identification of the damaged lung tissue. For example, there may be no need for imaging the lungs of the subject to identify regions or sites of damaged tissue before administering a composition of the invention to the subject. Preferably, the targeting moiety acts to direct the administered composition to sites of damaged lung tissue, for example, by virtue of higher amounts of damage-correlated moieties in areas of the lung affected by the pulmonary condition compared with areas of the lung that are not affected or that are affected to a lesser extent. For example, where an alpha-1 antitrypsin moiety is used as a targeting moiety, the alpha-1 antitrypsin moiety can recognize and bind to elastase, which is found in higher concentrations at sites of damaged lung tissue in certain pulmonary conditions, e.g., in emphysema.

Cross-linking of the damaged lung tissue can then bring about a reduction in lung volume, for example, by sealing and/or keeping collapsed regions of over-inflated lung tissue, preferably freeing up space for the expansion of remaining non-damaged or healthier tissue. In emphysema, for instance, regions of the lung that have lost elasticity required for exhalation can be collapsed and/or sealed by the methods described herein. Because the cross-linked tissue occupies a smaller volume than, e.g., the enlarged alveoli at sites of damaged tissue, methods of this invention can reduce lung volume overall. The present invention can thus provide a non-surgical, less-invasive and/or safer approach for achieving some of the benefits of lung volume reduction surgery. Further, targeting sites of damaged lung tissue allows for localized volume reduction, which in turn can minimize untoward side effects of lung volume reduction, such as exacerbating V/Q imbalance, changing arterial oxygenation, or triggering acute hypoxemia. Ingenito et al., (2002) Bronchoscopic Lung Volume Reduction Using tissue engineering principles, American Journal of Respiratory and Critical Care Medicine, Vol. 167 pgs 771-778. It is to be understood also that the methods of the present invention may be used in conjunction with a surgical procedure, such as LVRS and the use of knifeless staplers (see, e.g., Swanson et al., “No-cut thoracoscopic lung plication: A new technique for lung volume reduction surgery”, J Am Coll Surg Vol. 185 pgs 25-32 (1997)), as well as other approaches for treating pulmonary conditions, including use of coupled targeting moieties and/or imaging methods described herein.

Cross-linking of the cross-linkable moieties can be achieved by any methods known in the art and/or described herein. For example, a second composition may be administered that comprises a cross-linking activating moiety. “Cross-linking activating moiety” as used herein refers to any moiety that can bring about cross-linking between more than one cross-linkable moieties and/or that can form more than one bond with components (e.g. proteins) of damaged lung tissue. Preferably, a cross-linking activating moiety comprises a di- or polyfunctional group. For example, where the cross-linkable moiety is at least one of a hydroxyl group, a carboxyl group, an ester group, a cyano group, a thiol group (e.g., a cysteine group), a carbonyl group, an aldehyde group, a ketone group, a primary amine group, a secondary amine group, and/or a lysine group the cross-linking activating moiety may comprise a diol, a polyol, a dicarboxylic acid (e.g., fumaric, maleic, phthalic or terephthalic acid), a polycarboxylic acid, a diester, a polyester, a diamine and/or a polyamine. The di- or polyfunctional group can form covalent linkages with more than one cross-linkable moieties, preferably between cross-linkable moieties coupled to targeting moieties binding to damage-correlated moieties at different sites of damaged lung tissue, e.g., at different sites within an enlarged alveolus. Linkage may include, for example, amide formation (e.g., through the condensation of an amino group with an activated ester, such as, e.g., an NHS or sulfo-NHS ester), imine formation, carbodiimide condensation, disulfide bond formation, and/or use of a specific binding pair e.g., using a biotin-avidin interaction. The cross-linking can therefore serve to seal and/or keep collapsed air spaces at sites of damaged lung tissue, e.g., in areas of over-inflated alveoli, as characteristic of certain pulmonary conditions, including emphysema.

Di- and/or polyamines that may be used in the practice of this invention include aliphatic and/or aromatic di- and/or polyamines, as well as two or more aliphatic and/or aromatic monoamines suitably linked together. For example, monomeric, di- and/or polyamines that may be used in the practice of this invention can comprise aminopyrimidine, aniline, benzidine, diaminodiphenylamine, diphenylamine, hydrazine, hydrazide, toluene-diamine, and/or triethylenediamine. Di- and/or polyamines that may be used also can comprise, for example, acetamide, acrylamide, benzamide, cyanamide, and/or urea. Di- and/or polyalcohols that may be used include aromatic and/or aliphatic alcohols, including, for example, 1,4-butanediol, phenols, polyvinyl alcohols, and/or d-sorbitol. Examples of dicarbonyls that may be used in the practice of the present invention include dicarbonyls comprising acetate, e.g., α-haloacetate derivatives, acetylacetone, diethylmalonate, ethylacetone, malonamide, malonic acid and/or malonic esters or salts thereof. Other carbonyl groups that may be used include α, β-unsaturated carbonyl groups (e.g., maleimide) and/or α-halocarbonyl groups (e.g., iodoacetamide derivatives). Di- and/or polyfunctional ketones may also be used including, e.g., 2,5-hexanedione, and/or di- and/or polyfunctional ketones comprising two or more linked monofunctional ketones, such as cyclohexanone and/or cyclopentanone. Di- and/or polyfunctional aldehydes may also be used, see, e.g., U.S. Pat. No. 6,329,337 and/or U.S. Pat. No. 6,372,229. For example, at least one aldehyde selected from gelatin-resorcin-aldehyde, glyoxal, succinaldehyde, glutaraldehyde, malealdehyde, dextrandialdehyde, and saccharides oxidized by m-periodate may be used.

As will be appreciated by one skilled in the art, aldehydes and/or ketones described herein can exist as hydrates in aqueous solution, e.g., existing as hemi-acetals and/or hemi-ketals in aqueous solution. In preferred embodiments, such hydrates can revert back to the corresponding aldehyde and/or ketone for cross-linking. In some embodiments, hydrates of aldehydes and/or ketones and/or hydrates of other cross-linking activating moieties are themselves capable of bringing about cross-linking between more than one cross-linkable moieties and/or of forming more than one bond with components (e.g. proteins) of damaged lung tissue.

Other cross-linking activating moieties that may (or may not) be used in the practice of the present invention include a protein or a mixture of proteins (including synthetic peptides and/or recombinant proteins), such as collagen and/or albumin and/or lipoprotein along with other minor additives, optionally as well as hydrogel, polyglycolic acid, polylactic acid, polydioxanone, polytrimethylene carbonate, polycarprolactone, and/or glutaraldehyde, polyethylene glycol, polyethylene glycol disuccinimidoyl succinate, as well as polymerizable monomers, such as 1,1-disubstituted ethylene monomers or acetates, e.g., α-haloacetate, acrylate, acrylate glue, anhydrides cross-linked with polyols, cyano groups, e.g., cyanoacrylate, epoxy, gelatin resorcinol formaldehyde, gelatin resorcinol glutaraldehyde, hyaluronic acid cross-linked with hydrazines, photopolymerizable monomers, silicone, silicone rubber, starches, urethane, vinyl-terminated monomers, and/or any combination thereof. Other cross-linking activating moieties that may be used in the practice of the present invention include alkyl bis(2-cyanoacrylate), triallyl isocyanurate, alkylene diacrylate, alkylene dimethacrylate, and/or trimethylol propane triacrylate. Other cross-linking activating moieties that may be used in the practice of the present invention include disulfide, carbodiimide and hydrazine. Other suitable cross-linking activating moieties may be found in the art, for example, U.S. Pat. No. 3,940,362; U.S. Pat. No. 5,328,687; U.S. Pat. No. 3,527,841; U.S. Pat. No. 3,722,599; U.S. Pat. No. 3,995,641; and/or U.S. Pat. No. 5,583,114, each incorporated herein by reference. Still another cross-linking activating moiety that may be used includes a product formed by reacting glutaraldehyde with amino acids and/or peptides, as described in U.S. Pat. No. 6,310,036. Cross-linkable and/or cross-linking activating moieties may also include suitable monomers disclosed in U.S. Publication No. 2002/0147462, such as, for instance, monomeric n-butyl-2-cyanoacrylate (Eng et al., “Successful closure of bronchopleural fistula with adhesive tissue”, Scand J Thor Cardiovasc Surg, Vol. 24 pgs 157-59 (1990) and Inaspettato et al., “Endoscopic treatment of bronchopleural fistulas using n-butyl-2-cyanoacrylate”, Surgical Laparoscopy &Endoscopy, Vol. 4 No. 1 pgs 62-64 (1994)).

The choice of cross-linking activating moiety can depend, at least in part, on the cross-linkable moieties used. Where the cross-linkable moiety is fibrin and/or fibrinogen, the cross-linking activating moiety may comprise a fibrin activator and/or a fibrinogen activator. For example, thrombin, a thrombin receptor agonist, batroxobin, and/or calcium can be used to initiate cross-linking of fibrinogen. It is also to be understood that any combination of cross-linking activating moieties may be used, depending on, for example, the combination of cross-linkable moieties administered. Further, some embodiments provide a composition comprising a targeting moiety coupled to a cross-linking activating moiety, e.g., to facilitate migration and/or distribution of the cross-linking activating moiety to sites of damaged lung tissue. Those of skill in the art will recognize other suitable cross-linking activating moieties that may be used in the practice of the instant invention, including, for example, any biocompatible cross-linking activating moiety that can form a biocompatible cross-linked product with a cross-linkable moiety used. In still more preferred embodiments, the cross-linkable and cross-linking activating moieties used are medically acceptable and form medically acceptable cross-links.

In some embodiments, one or more of the cross-linkable, targeting and/or cross-linking activating moieties are thermally stabilized. That is, the moiety may be modified, adapted and/or otherwise engineered to withstand heat, e.g., heat generated by a cross-linking reaction within lung tissue of a subject. For example, heat-stabilized glutaraldehyde in an aqueous carrier may be used, and in some embodiments amino acid modifications in protein targeting moieties may confer increased thermal stability.

The cross-linkable and cross-linking activating moieties can be added in appropriate ratios to facilitate cross-linking. The ratio to be used may depend on the cross-linkable and/or cross-linking activating moieties used, the rate of cross-linking desired, and/or other reaction conditions appreciated by those of skill in the art. For example, a ratio of at least about 1:1; at least about 1:2, at least about 1:5, at least about 1:10; at least about 1:15, or at least about 1:20 may be used.

It will be recognized by those of skill in the art that certain of these cross-linking activating moieties may be suitable for use alone, i.e., without a corresponding cross-linkable moiety. For example, biotin groups, amine groups, carboxylic acid groups, cyanate groups (e.g. isothiocyanate), thiol groups, disulfide groups, cyano groups (e.g., α-halocarbonyl groups, α, β-unsaturated carbonyl groups), an acetate group (e.g., α-haloacetate group), hydrazine groups, cyanoacrylate, acrylic glue, and/or silicone moieties, as well as bifunctional linkers, may be used to bring about crosslinking of damaged lung tissue without the use of a separate cross-linkable moiety. Further, various combinations of cross-linking activating moieties may be used, administered together at the same time or separately at different times of administration. For instance, a dipolyaldehyde and/or polyaldehyde may be combined with a mixture of proteins, such as albumin and/or collagen, and optionally other minor additives. Also, as mentioned above, the cross-linking activating moiety may in some embodiments be coupled to a targeting moiety, for example, to an alpha-1 antitrypsin molecule, fragment thereof, and/or derivative thereof; or to a combination of targeting moieties, including, for example, any combination of types of targeting moieties provided herein.

It is also to be understood that some embodiments would not require a cross-linking activating moiety for initiation of cross-linking. For example, if fibrin is used as the cross-linkable moiety, e.g., a fibrin monomer, such as fibrin I monomers, fibrin II monomers and/or des BB fibrin monomers, the monomers may spontaneously cross-link. For instance, fibrin I monomers may cross-link upon contacting a subject's blood, which contains thrombin and factor XII.

Various types of cross-linking reactions may be used in the practice of the present invention including, for example, free radical reactions, cross-linking by zwitterions and/or ion pairs, anions and/or cations. See e.g., U.S. Pat. Nos. 6,010,714; 5,582,834; 5,575,997; 5,514,372; 5,514,371 and 5,328,687 and 5,981,621. Cross-linking reactions of the present invention may also involve amide formation, imine formation, carbodiimide condensation, disulfide bond formation, and use of a specific binding pair, e.g., using a biotin-avidin interaction.

In some preferred embodiments, the method for reducing lung volume does not damage epithelial cells within lung tissues, e.g., it may not cause scar tissue formation, and/or may not cause fibroblast proliferation, and/or may not cause collagen synthesis. In some preferred embodiments, the methods cross-link and/or seal sites of damaged lung tissue within an alveolus, more preferably within an enlarged alveolus distal to a terminal bronchiole. In some preferred embodiments, the methods of the present invention do not cause occlusion of a lumen of a bronchial tube of a lung of the subject. Without being limited to a particular mechanism, methods of the present invention can reduce lung volume by keeping cross-linked and/or sealed enlarged air spaces, rather than by (mechanically) attempting to block air-flow to damaged lung tissue. That is, in preferred embodiments, cross-linking serves to keep collapsed and/or sealed blind ending sacs, rather than there being any or any substantial amount of lung tissue distal to the cross-linked sites.

In some preferred embodiments, the method for reducing lung volume can involve damage to lung tissue. For example, in some embodiments a sclerosing agent can be used as part of the administered composition, for instance, a sclerosing agent may be coupled to a targeting moiety of the present invention. In some embodiments, the sclerosing agent may be administered alone; or it may be administered separately at the same time as, before, or after administration of targeting, cross-linkable, and/or cross-linking activating moieties of the present invention. The sclerosing agent can serve to bring about scar tissue formation, and/or fibroblast proliferation, and/or collagen synthesis, facilitating sealing of regions of damaged lung tissue. Sclerosing agents that may be used in the present invention include Doxycycline, Bleomycin, Minocycline, Doxorubicin, Cisplatin+Cytarabine, Mitoxantrone, Corynebacterium Parvum, Streptokinase, Urokinase, and the like. Other agents and/or methods for damaging lung tissue may also be used in the practice of the present invention, optionally along with components of the extracellular matrix e.g., hyaluronic acid. See e.g., U.S. Publication No. 2004/0047855.

In yet still preferred embodiments, the cross-linking methods of the present invention can be carried out without the use of a catheter, and/or without the use of an endotracheal applicator, and/or without the use of bronchoscopy (e.g., without the use of a bronchoscope), and/or without the use of laproscopy, and/or without the use of open surgery, e.g., thracotomy.

In some embodiments, cross-linking activating moieties are administered after allowing sufficient time for targeting of the administered cross-linkable moieties to sites of damaged lung tissue. In preferred embodiments, the targeting moiety recognizes and binds its damage-correlated moiety in at least about 30 seconds, at least about 1 minute, at least about 3 minutes, or at least about 5 minutes. In preferred embodiments, the targeting moiety recognizes and binds its damaged-correlated moiety in less than about 3 hours, in less than about 2 hours, in less than about 1 hour, in less than about 45 minutes, in less than about 30 minutes, in less than about 20 minutes, or in less than about 10 minutes. Also, in some embodiments, unbound targeting moiety may be removed from the lungs, e.g., by lavage and/or washing (e.g., with saline) and/or by collapsing, before administration of cross-linking activating moiety.

Cross-linking may be facilitated by deflating and/or collapsing a first portion or all of the lung of the subject. Such deflating and/or collapsing can be achieved by any techniques known in the art or herein disclosed. For example, the collapsing may involve the use of negative pressure from within the lung and/or positive pressure from without the lung. Also, in some embodiments, a preparation to induce and/or facilitate collapse may be used, e.g., a physiologically acceptable solution containing an anti-surfactant, such as an agent that can increase surface tension of fluids lining alveoli. For example, an anti-surfactant may be administered prior to, during, and/or after administration of the composition comprising the cross-linkable moiety and/or the cross-linking activating moiety. For instance, fribrin and/or fibrinogen may be used, which can act both as an anti-surfactant as well as aiding cross-linking.

Other suitable surfactants that may be used to facilitate cross-linking include Triton x-100, beractant, colfosceril, and/or palmitate; anionic surfactants such as sodium tetradecyl sulfate; cationic surfactants such as tetrabutylammonium bromide and/or butyrylcholine chloride; nonionic surfactants such as polysorbate 20 (e.g., Tween 20), polysorbate 80 (e.g. Tween 80), and/or poloxamers; amphoteric and/or zwitterionic surfactants such as dodecyldimethyl(3-sulfopropyl)ammonium hydroxide, inner salt; amines, imines and/or amides, such as arginine, imidazole, povidine, tryptamine, and/or urea; alcohols such as ascorbic acid, ethylene glycol, methyl gallate, tannins and/or tannic acid; phosphines, phosphites and phosphonium salts, such as triphenylphosphine and/or triethyl phosphite; inorganic bases and/or salts, such as calcium sulfate, magnesium hydroxide, sodium silicate, and/or sodium bisulfite; sulfur compounds such as polysulfides and/or thiourea; polymeric cyclic ethers such as calixarenes, crown ethers, monensin, nonactin, and/or polymeric epoxides; cyclic and acyclic carbonates; organometallics (e.g., naphthenate and manganese acetylacetonate); phase transfer catalysts (e.g., Aliquat 336); and radical initiators and radicals (e.g., di-t-butyl peroxide and/or azobisisobutyronitrile).

Cross-linking may also be facilitated by filling the lung or a portion thereof with an absorbable gas, such as oxygen, e.g., to promote atelectasis. Ingenito et al., “Bronchoscope volume reduction—A safe and effective alternative to surgical therapy for emphysema,” American Journal of Respiratory and Critical Care Medicine, Vol 164 pgs 295-301 (2001).

In some embodiments, a lavage of saline may be used to reduce the amount of surfactant naturally occurring in the lungs. Cross-linking may also be facilitated by use of a lavage capable of removing, e.g., any other moieties that may impede, reduce and/or otherwise interfere with targeting. For example, in some embodiments, cross-linking may be facilitated by use of an anti-secretory agent that hinders and/or prevents mucous secretion in the lung or a portion thereof. For example, the anti-secretory agent may be administered prior to, during, and/or after administration of the composition comprising the cross-linkable moiety, the cross-linking activating moiety, and/or other moiety and/or agent. Examples of anti-secretory agents that may be used include, for example, anticholinergic moieties, atronie, and/or atropinic moieties. Removal of mucous or excessive mucous from the lung, preferably from enlarged alveoli distal to terminal bronchioles, e.g., by washing, can also facilitate cross-linking and/or binding of the targeting moiety to its damage-correlated moiety. Adhesion of a composition of the present invention to a mucous-coated wall within a bronchus, bronchiole, or alveolus can be facilitated by virtue of targeting moieties of the present invention binding to their respective damage-correlated moieties and, for example, reducing and/or avoiding slippage.

In some embodiments, mechanical force may be used externally to push one area of the lung closer to another, for example, to help collapse and/or deflate an enlarged air space. A portion of a lobe of the lung may be pressed externally using, for example, a balloon, air pressure, manual pressure, and/or an instrument such as a paddle, a net, a strap that can be synched up, or magnets. In some embodiments, such pressure is applied to two or more sides of a lung lobe simultaneously. For example, endoscopes and/or magnetic probes can be used to apply local pressure (applenate) to more than one side.

In some embodiments, a first portion or all of the lung may be drawn together from the inside using, for example, a cable and hook to grab and pull tissue, for instance, towards the user. Other devices that can be used include graspers, such as an expanding grasper assembly that can be sheathed; and/or anchors that can be left behind, for example, by being uncoupled from a cable or wire after lung tissue has been drawn together. In some embodiments, magnetic probes can be placed at different locations within the lung where the probes attract one another, thereby attracting one region of the lung to the other, e.g., one bronchi to another. Additionally, mechanical force may be used to change the shape of such devices after insertion, such as by using a core wire or activating a NiTi device after placement. In still other embodiments, the lungs or a first portion thereof are deflated trans-thoracically. Other methods and/or devices known in the art to facilitate lung deflation and/or collapse may also be employed, e.g. see U.S. Publication No. 2003/0070682.

Such deflating and/or collapsing is preferably carried out after allowing sufficient time for distribution of the administered cross-linking activating moieties to areas of damaged lung tissue. In some embodiments, for example, deflating and/or collapsing is carried out approximately 2 to approximately 3 minutes after administration of the cross-linking activating moieties. Also, the lung, or a first portion thereof, is preferably allowed to remain in a collapsed and/or deflated state for a time sufficient to permit cross-linking to take place. Depending on the composition used, e.g., the targeting moieties used, the lung or a first portion thereof can be kept deflated and/or collapsed for at least approximately 3 days, at least approximately 2 days (48 hours), at least approximately 24 hours, at least approximately 12 hours, at least approximately 5 hours, at least approximately 1 hour, at least approximately 45 minutes, at least approximately 20 minutes, at least approximately 10 minutes, at least approximately 5 minutes, at least approximately 2 minutes, at least approximately 1 minute, at least approximately 30 seconds, or at least approximately 15 seconds. In some embodiments, the lung or a first portion thereof can be kept deflated and/or collapsed for less than about 30 minutes, less than about 20 minutes, less than about 10 minutes, or less than about 8 minutes.

In some embodiments, a catalytic amount of a rate modifier may be added to modify the rate of the cross-linking reaction. For example, various set or cure times may be used, where the cross-linking reaction occurs in at least about 20 seconds, at least about 30 seconds, at least about 1 minute, at least about 90 seconds, at least about 2 minutes, at least about 150 seconds, at least about 3 minutes, at least about 4 minutes, at least about 5 minutes, at least about 6 minutes, at least about 10 minutes, or at least about 15 minutes. The cross-linking reaction may occur in less than about 20 minutes, in less than about 25 minutes, in less than about 30 minutes, in less than about 1 hour, in less than about 2 hours, or in less than about 3 hours. Cure times may be tailored by use of various techniques known in the art, for example, by using buffers having different pH values.

A second portion of the lung can then be re-inflated, where the second portion comprises part, but preferably not all, of the first portion or all of the lung that was deflated and/or collapsed. In preferred embodiments, this second portion does not comprise at least some damaged lung tissue, which remains collapsed and/or sealed by virtue of the cross-linking. The cross-linking preferably forms a stable mesh that keeps the collapsed region from re-inflating. In more preferred embodiments, the majority of damaged lung tissue remains cross-linked and/or collapsed, while the majority of non-damaged lung tissue is left in a functional condition. For example, at least about 60%, at least about 80%, and most preferably at least about 90% of damaged lung tissues is cross-linked; while less than about 40%, less than about 20%, and most preferably less than about 10% of non-damaged lung tissue remains not cross-linked. Reduction in overall lung volume improves mechanical function, e.g., mechanical functioning of healthier and/or more elastic tissue.

In preferred embodiments, cross-linking results in at least about a 0.5% overall lung volume reduction, at least about a 1% overall lung volume reduction, at least about a 1.5% overall lung volume reduction, at least about a 2% overall lung volume reduction, at least about a 3% overall lung volume reduction, at least about a 4% overall lung volume reduction, at least about a 5% overall lung volume reduction, or at least about a 10% overall lung volume reduction. In preferred embodiments, cross-linking results in less than about a 40%, less than about a 35%, less than about a 30%, less than about a 25%, less than about a 20%, or less than about a 15% overall lung volume reduction. Such reduction may be achieved upon a single or multiple administrations of compositions of the present invention. A reduction of about 2% to about 3% overall lung volume reduction can be expected to produce a beneficial effect in a subject receiving such treatment, e.g., at least to a similar extent as that produced in LVRS.

Also in preferred embodiments, the cross-linking is permanent, or at least semi-permanent, for a period of time between successive treatments as described herein, e.g., resisting biodegradation (e.g., hydrolysis) for the period of time between administrations of a composition of the present invention. In certain embodiments, at least about 70%, at least about 80%, at least about 90%, or at least about 98% of the cross-links remain intact for a period of time. In some preferred embodiments, the period is at least about one month, at least about 2 months, at least about 3 months, at least about 6 months, at least about a year, at least about 2 years, at least about 3 years, at least about 5 years, or at least about 10 years. In some preferred embodiments, the period is less than about 50 years, less than about 30 years, less than about 20 years, or less than about 15 years. In most preferred embodiments, the cross-linking keeps some damaged lung tissue collapsed and/or sealed for the remainder of the life of the subject, for example, resisting biodegradation indefinitely.

One of skill in the art will recognized that the permanence and/or biodegradability of the cross-links can depend on the cross-linkable moiety, the cross-linking activating moiety and/or the conditions of cross-linking and/or other agents and/or moieties used, and can be controlled accordingly, e.g., by techniques known the art and/or disclosed herein.

In preferred embodiments, some or all of the cross-links are strong enough to withstand mechanical pressures experienced within the lung. For example, the strain range corresponding to functional residual capacity during normal breathing does not result in breakage of at least about 30%, at least about 40%, at least about 50%, at least about 60%, at least about 70%, at least about 80%, at least about 90%, or at least about 95% of the cross-links in some preferred embodiments.

In some preferred embodiments, the cross-links exhibit a tear strength of at least about 50 g/sq. cm, at least about 100 g/sq. cm, at least about 200 g/sq. cm, or at least about 300 g/sq. cm. In some preferred embodiments, the cross-links exhibit a tear strength of less than about 5,000 g/sq. cm, less than about 3,000 g/sq. cm, less than about 1500 g/sq. cm, less than about 1300 g/sq. cm, less than about 1200 g/sq. cm, less than about 1000 g/sq. cm, less than about 800 g/sq. cm, less than about 600 g/sq. cm, or less than about 400 g/sq. cm.

Similarly, in preferred embodiments, the binding interaction between a targeting moiety and its corresponding damage-correlated moiety is permanent, or at least semi-permanent, for a period of time between successive treatments as described herein, e.g., binding irreversibly, substantially irreversibly, or at least with a high binding constant, e.g., to resist dissociation for the period of time between administrations of a composition of the present invention. For example, an alpha-1 antitrypsin moiety may form a pseudo-irreversible equimolar complex with neutrophil elastase in some embodiments. See, e.g., Sifers et al., “Genetic Control of Human Alpha-1 Antitrypsin”, Mol. Biol. Med., Vol. 6 pgs. 127-135 (1989). Without being limited to a particular theory or mode of action, the alpha-1 antitrypsin moiety may form an acyl-enzyme complex with its target. In some embodiments, binding can be further enhanced by genetic modification or by shuffling of known binding domains. As another example, a serpin moiety may react with its target protease to form a sodium dodecyl sulfate(SDS)-stable equimolar complex. Without being limited to a particular theory or mode of action, the complex between a serpin and its target protease may involve a covalent ester bond linkage, where an active site Serine residue of the protease binds a C-terminal residue of a cleaved form of the serpin to form an acyl-enzyme complex. See, e.g., U.S. Publication No. 2003/0216321. As yet another example, a monocyte elastase inhibitor moiety can form a covalent complex and/or an essentially irreversible complex with elastase. See, e.g., International Publication WO 96/10418 and U.S. Pat. No. 5,827,672.

In certain embodiments, at least about 70%, at least about 80%, at least about 90%, or at least about 98% of the targeting moieties remain bound to corresponding damage-correlated moieties for a period of time. In some preferred embodiments, the period is at least about one month, at least about 2 months, at least about 3 months, at least about 6 months, at least about a year, at least about 2 years, at least about 3 years, at least about 5 years, or at least about 10 years. In some preferred embodiments, the period is less than about 50 years, less than about 30 years, less than about 20 years, or less than about 15 years. In most preferred embodiments, the binding keeps some damaged lung tissue collapsed and/or sealed for the remainder of the life of the subject, for example, resisting dissociation indefinitely.

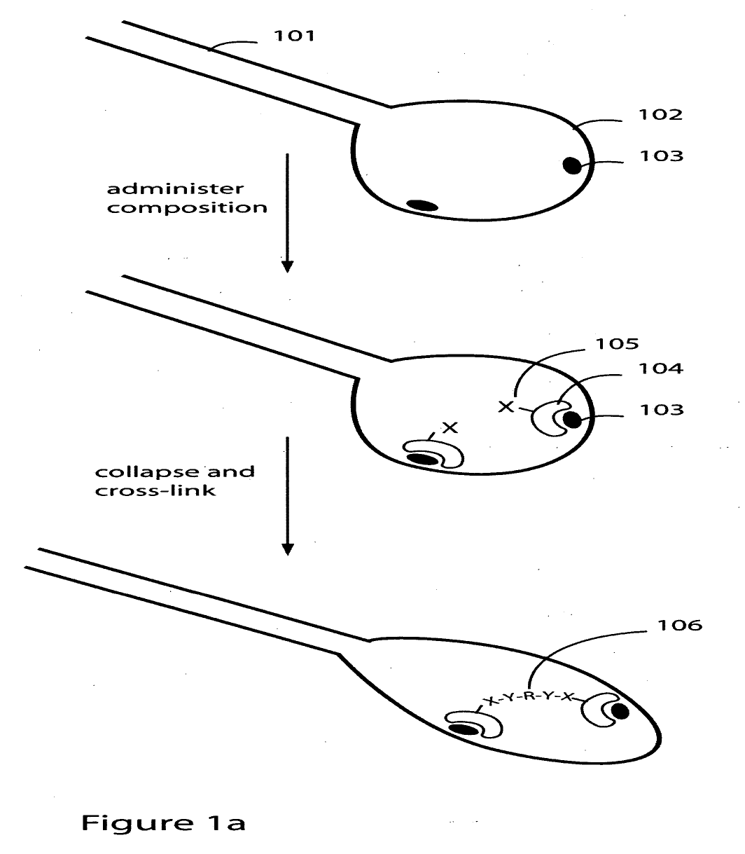

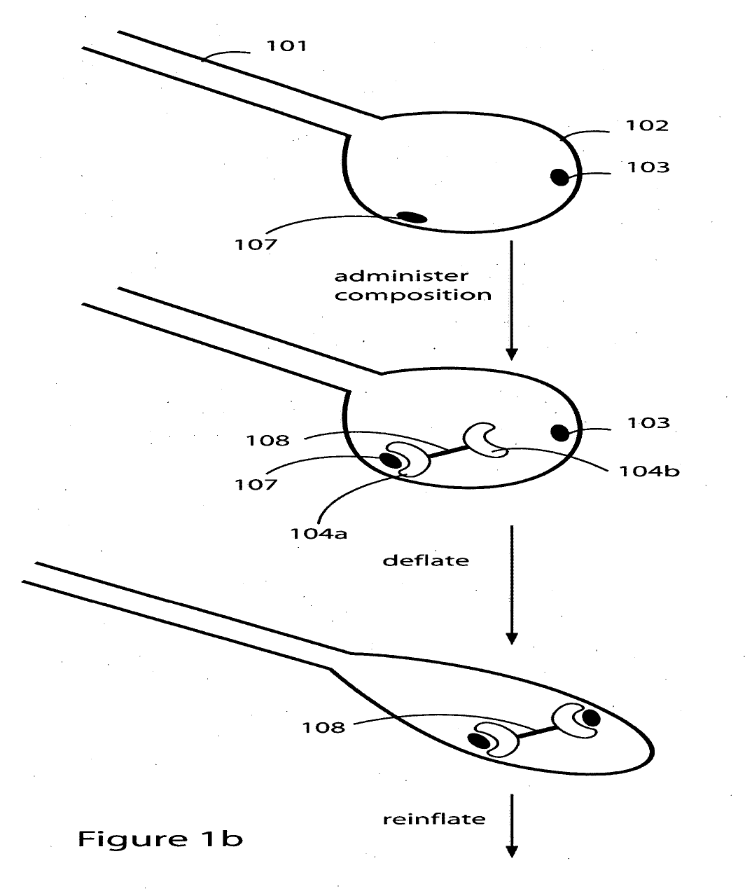

FIG. 1a illustrates one embodiment of a method to reduce lung volume using a composition comprising a cross-linkable moiety coupled to a targeting moiety that targets damaged lung tissue. This figure provides an overview only, and is in no way intended to be limiting with respect to the present invention. For example, those skilled in the art will readily appreciate variations and modifications of the scheme illustrated. The figure schematically illustrates a terminal bronchiole 101 terminating in an airspace of an alveolus 102. As mentioned above, the air space may be over-inflated and/or enlarged in certain pulmonary conditions, such as emphysema. Within the walls of the airspace, high amounts of damage-correlated moieties 103 are found, for example, at sites of damaged lung tissue and/or within the epithelial lining fluid.

A composition of the invention is administered, where the composition comprises a targeting moiety 104 that targets damaged lung tissue, for example, by recognizing and binding its target damage-correlated moiety 103. In the illustrated embodiment, the composition also comprises a cross-linkable moiety (X) 105 coupled to the targeting moiety 104. FIG. 1a illustrates how different targeting moieties recognize and bind damage-correlated moieties at various sites within the air space.

Following cross-linking, the cross-linkable moieties 105 become cross-linked, for example, via a cross-linking activating moiety 106. The cross-linking activating moiety 106 may comprise a di-functional group, depicted in the figure as -Y-R-Y-, where Y represents a group capable of coupling to the cross-linkable moieties (X) 105, e.g., to form covalent linkages between two cross-linkable moieties, and R represents a linking moiety between the Y groups, for example, but not limited to, an aliphatic chain. The cross-linking activating moiety 106 couples the cross-linkable moieties 105 that are themselves coupled to targeting moieties 104 bound to damage-correlated moieties 103 found at various sites within the air space. FIG. 1a illustrates how, following collapse, cross-linking can keep the walls of the air space closer together and thereby reduce lung volume.

The methods of reducing lung volume described herein find use in the treatment of a number of pulmonary conditions in animal subjects. The term “animal subject” as used herein includes humans as well as other mammals. The term “treating” as used herein includes achieving a therapeutic benefit and/or a prophylactic benefit. By therapeutic benefit is meant eradication or amelioration of the underlying pulmonary condition being treated. For example, in an emphysematous patient, therapeutic benefit includes eradication or amelioration of the underlying emphysema, including improved lung function, exercise capacity, quality of life, and reduced hospitalization. Also, a therapeutic benefit is achieved with the eradication or amelioration of one or more of the physiological symptoms associated with the underlying pulmonary condition such that an improvement is observed in the subject, notwithstanding the fact that the subject may still be afflicted with the pulmonary condition. For example, with respect to emphysema, administration of compositions of the invention can provide therapeutic benefit not only when areas lacking elasticity are collapsed, but also when an improvement is observed in the subject with respect to other disorders that accompany emphysema like chronic pulmonary infection. For example, addition of targeting moieties comprising protease inhibitors may ameliorate emphysema by reducing protease activity, e.g., as described in the art. For prophylactic benefit, a composition of the present invention may be administered to a subject at risk of developing a pulmonary condition, for example, emphysema, or to a subject reporting one or more of the physiological symptoms of such a condition, even though a diagnosis may not have been made.

Another aspect of the present invention provides a method of reducing lung volume comprising administering to a subject in need thereof a composition comprising a first targeting moiety and a second targeting moiety wherein said targeting moieties are coupled and wherein said targeting moieties target damaged lung tissue; and allowing said targeting moieties to target different sites of damaged lung tissue, thereby reducing lung volume. In preferred embodiments, the different sites comprise different sites within an enlarged air space, e.g., within alveolar walls of an over-inflated alveolus distal to a terminal bronchiole, as characteristic of some pulmonary conditions, including emphysema. For example, the first targeting moiety can target a first damage-correlated moiety while the second targeting moiety can target a second damage-correlated moiety, where the first and second damage-correlated moieties occur at different sites. As the coupled targeting moieties bind to different sites within an air space, following deflation and/or collapse, the coupled targeting moieties can act to keep different sites closer together, thereby keeping the air space in a collapsed and/or sealed state. Also, as the targeting moieties recognize and/or bind damage-correlated moieties found in higher amounts in areas of the lung affected by a pulmonary condition compared with areas of the lung that are not affected or that are affected to a lesser extent, regions of damaged lung tissue can be selectively and/or preferentially collapsed and/or sealed, preferably freeing up space for the expansion of remaining non-damaged or healthier tissue.

In preferred embodiments, the method utilizing coupled targeting moieties can be performed without prior identification of the damaged lung tissue. For example, there may be no need for imaging the lungs of the subject to identify regions of damaged tissue before administering a composition of the invention to the subject. The targeting moiety acts to direct the administered composition to sites of damaged lung tissue, for example, by virtue of higher amounts of damage-correlated moieties in areas of the lung affected by the pulmonary condition compared with areas of the lung that are not affected or that are affected to a lesser extent. For example, where an alpha-1 antitrypsin moiety is used as a targeting moiety, the alpha-1 antitrypsin moiety can recognize and bind to elastase, which is found in higher concentrations at sites of damaged lung tissue in certain pulmonary conditions, e.g., in emphysema.

Because the collapsed tissue occupies a smaller volume than the enlarged alveoli at sites of damaged tissue, methods of this invention can reduce lung volume overall. The present invention can thus provide a non-surgical, less-invasive and/or safer approach for achieving at least some of the benefits of lung volume reduction surgery. Further, targeting of sites of damaged lung tissue allows localized volume reduction, which in turn minimizes untoward side effects, such as exacerbating V/Q imbalance, changing arterial oxygenation, or triggering acute hypoxemia. Ingenito et al., “Bronchoiscopic Lung Volume Reduction Using tissue engineering principles”, American Journal of Respiratory and Critical Care Medicine, Vol. 167 pgs. 771-778 (2002). It is to be understood also that the methods of the present invention may be used in conjunction with a surgical procedure, such as LVRS, as well as other approaches for treating pulmonary conditions, including cross-linking and/or imaging methods described herein, and/or other methods described in any of entitled “Targeting Damaged Lung Tissue Using Compositions,” filed Dec. 8, 2004; “Targeting Damaged Lung Tissue,” filed Dec. 8, 2004; “Targeting Sites of Damaged Lung Tissue Using Composition,” filed Dec. 8, 2004; “Targeting Sites of Damaged Lung Tissue,” filed Dec. 8, 2004; “Imaging Damaged Lung Tissue Using Compositions,” filed Dec. 8, 2004; “Glue Compositions for Lung Volume Reduction,” filed Dec. 8, 2004; “Lung Volume Reduction Using Glue Compositions,” filed Dec. 8, 2004; “Glue Composition for Lung Volume Reduction,” filed Dec. 8, 2004; and “Lung Volume Reduction Using Glue Composition,” filed Dec. 8, 2004, each of which is herein incorporated in its entirely.

Further, in some preferred embodiments, the method for reducing lung volume does not damage epithelial cells within lung tissues and, e.g., it may not cause scar tissue formation, and/or may not cause fibroblast proliferation, and/or may not cause collagen synthesis. In some preferred embodiments, the methods seal and/or keep collapsed sites of damaged lung tissue within an alveolus, more preferably within an enlarged alveolus distal to a terminal bronchiole. In some preferred embodiments, the methods of the present invention do not cause occlusion of a lumen of a bronchial tube of a lung of the subject. Without being limited to a particular mechanism, methods of the present invention can reduce lung volume by sealing enlarged air spaces, rather than by (mechanically) attempting to block air-flow to damaged lung tissue. That is, in preferred embodiments, targeting of different sites of damaged lung tissue by coupled targeting moieties serves to seal and/or keep collapsed blind ending sacs, rather than there being any or any substantial amount of lung tissue distal to the collapsed regions. In yet still preferred embodiments, the lung volume reducing methods of the present invention can be carried out without the use of a catheter, and/or without the use of an endotracheal applicator, and/or without the use of bronchoscopy (e.g., without the use of a bronchoscope), and/or without the use of laproscopy, and/or without the use of open surgery, e.g., thoracotomy.

In some preferred embodiments, the method for reducing lung volume can involve damage to lung tissue. For example, in some embodiments a sclerosing agent can be used as part of the administered composition of the present invention, for instance, a sclerosing agent may be coupled to a targeting moiety of the present invention. In some embodiments, the sclerosing agent may be administered alone; or it may be administered separately at the same time as, before, or after administration of targeting moieties of the present invention. The sclerosing agent can serve to bring about scar tissue formation, and/or fibroblast proliferation, and/or collagen synthesis, facilitating sealing of regions of damaged lung tissue. Sclerosing agents that may be used in the present invention include Doxycycline, Bleomycin, Minocycline, Doxorubicin, Cisplatin+Cytarabine, Mitoxantrone, Corynebacterium Parvum, Streptokinase, Urokinase, and the like. Other agents and/or methods for damaging lung tissue may also be used in the practice of the present invention, optionally along with components of the extracellular matrix, e.g., hyaluronic acid. See e.g., U.S. Publication No. 2004/0047855.

Collapse of lung tissue, e.g., collapse of an enlarged air spaces within which a composition of the present invention is bound, may involve deflating and/or collapsing a first portion or all of the lung of the subject. Such collapsing can be achieved by any techniques known in the art or herein disclosed. For example, the deflating and/or collapsing may involve the use of negative pressure from within the lung and/or positive pressure from without the lung. Also, in some embodiments, a preparation to induce and/or facilitate deflation and/or collapse may be used, e.g., a physiologically acceptable solution containing an anti-surfactant, such as an agent that can increase surface tension of fluids lining alveoli. For example, an anti-surfactant may be administered prior to, during, and/or after administration of the composition comprising coupled targeting moieties. For instance, fribrin and/or fibrinogen may be used. In some embodiments, a lavage of saline may be used to reduce the amount of surfactant naturally occurring in the lungs. Other suitable surfactants that may be used to facilitate collapse and/or deflation include Triton x-100, beractant, colfosceril, and/or palmitate; anionic surfactants such as sodium tetradecyl sulfate; cationic surfactants such as tetrabutylammonium bromide and/or butyrylcholine chloride; nonionic surfactants such as polysorbate 20 (e.g., Tween 20), polysorbate 80 (e.g. Tween 80), and/or poloxamers; amphoteric and/or zwitterionic surfactants such as dodecyldimethyl(3-sulfopropyl)ammonium hydroxide, inner salt; amines, imines and/or amides, such as arginine, imidazole, povidine, tryptamine, and/or urea; alcohols such as ascorbic acid, ethylene glycol, methyl gallate, tannins and/or tannic acid; phosphines, phosphites and phosphonium salts, such as triphenylphosphine and/or triethyl phosphite; inorganic bases and/or salts, such as calcium sulfate, magnesium hydroxide, sodium silicate, and/or sodium bisulfite; sulfur compounds such as polysulfides and/or thiourea; polymeric cyclic ethers such as calixarenes, crown ethers, monensin, nonactin, and/or polymeric epoxides; cyclic and acyclic carbonates; organometallics (e.g., naphthenate and manganese acetylacetonate); phase transfer catalysts (e.g., Aliquat 336); and radical initiators and radicals (e.g., di-t-butyl peroxide and/or azobisisobutyronitrile).

Deflation and/or collapse may also be facilitated by use of a lavage capable of removing any other moieties that may impede, reduce and/or otherwise interfere with targeting. For example, in some embodiments, cross-linking may be facilitated by use of an anti-secretory agent that hinders and/or prevents mucous secretion in the lung or a portion thereof. For example, the anti-secretory agent may be administered prior to, during, and/or after administration of the composition comprising coupled targeting moieties. Examples of anti-secretory agents that may be used include, for example, anticholinergic moieties, atronie, and/or atropinic moieties. Removal of mucous or excessive mucous from the lung, preferably from enlarged alveoli distal to terminal bronchioles, e.g., by washing, can also facilitate binding of the coupled targeting moieties to their respective damage correlated moieties. Adhesion of a composition of the present invention to a mucous-coated wall within a bronchus, bronchiole, or alveolus can be facilitated by virtue of targeting moieties of the present invention binding to their respective damage-correlated moieties, and, for example, reducing and/or avoiding slippage.

In some embodiments, mechanical force may be used externally to push one area of the lung closer to another, for example, to help collapse an enlarged air space. A portion of a lobe of the lung may be pressed externally using, for example, a balloon, air pressure, manual pressure, and/or an instrument such as a paddle, a net, a strap that can be synched up, or magnets. In some embodiments, such pressure is applied to two or more sides of a lung lobe simultaneously. For example, endoscopes and/or magnetic probes can be used to apply local pressure (applenate) to more than one side.

In some embodiments, a first portion or all of the lung may be drawn together from the inside using, for example, a cable and hook to grab and pull tissue, for instance, towards the user. Other devices that can be used include graspers, such as an expanding grasper assembly that can be sheathed; and/or anchors that can be left behind, for example, by being uncoupled from a cable or wire after lung tissue has been drawn together. In some embodiments, magnetic probes can be placed at different locations within the lung where the probes attract one another, thereby attracting one region of the lung to the other, e.g., one bronchi to another. Additionally, mechanical force may be used to change the shape of devices after insertion, such as by using a core wire or activating a NiTi device after placement. In still other embodiments, the lungs or a first portion thereof are deflated trans-thoracically. Other methods and/or devices known in the art to facilitate lung collapse may also be employed, e.g. see U.S. Publication No. 2003/0070682.

Such deflation and/or collapsing is preferably carried out after allowing sufficient time for distribution of the administered coupled targeting moieties to sites of damaged lung tissue. In some embodiments, for example, deflation and/or collapse is carried out approximately 2 to approximately 3 minutes after administration of a composition of the present invention. Also, the lung, or a first portion thereof, is preferably allowed to remain in a deflated and/or collapsed state for a time sufficient to permit recognition and/or binding of more than one of the coupled targeting moieties to corresponding damage-correlated moieties at different sites of damaged lung tissue. Depending on the type or types of targeting moieties used, the lung or a first portion thereof can be kept deflated and/or collapsed for at least approximately 3 days, at least approximately 2 days (48 hours), at least approximately 24 hours, at least approximately 12 hours, at least approximately 5 hours, at least approximately 1 hour, at least approximately 45 minutes, at least approximately 20 minutes, at least approximately 10 minutes, at least approximately 5 minutes, at least approximately 2 minutes, at least approximately 1 minute, at least approximately 30 seconds, or at least approximately 15 seconds. In some embodiments, the lung or a first portion thereof can be kept deflated and/or collapsed for less than about 30 minutes, less than about 20 minutes, less than about 10 minutes, or less than about 8 minutes.

A second portion of the lung can then be re-inflated, where the second portion comprises part, but preferably not all, of the first portion or all of the lung that was deflated and/or collapsed. In preferred embodiments, this second portion does not comprise at least some damaged lung tissue, which remains collapsed and/or sealed by virtue of coupled targeting moieties bound to different sites of damaged lung tissue. The binding preferably keeps the collapsed region from re-inflating. In more preferred embodiments, the majority of damaged lung tissue remains collapsed and/or sealed, while the majority of non-damaged lung tissue is left in a functional condition. For example, at least about 60%, at least about 80%, and most preferably at least about 90% of damaged lung tissues is collapsed; while less than about 40%, less than about 20%, and most preferably less than about 10% of non-damaged lung tissue is not and/or re-inflates. Reduction in overall lung volume improves mechanical function, e.g., mechanical functioning of healthier and/or more elastic tissue.

In preferred embodiments, binding of coupled targeting moieties results in at least about a 0.5% overall lung volume reduction, at least about a 1% overall lung volume reduction, at least about a 1.5% overall lung volume reduction, at least about a 2% overall lung volume reduction, at least about a 3% overall lung volume reduction, at least about a 4% overall lung volume reduction, at least about a 5% overall lung volume reduction, at least about a 10% overall lung volume reduction. In preferred embodiments binding of coupled targeting moieties results in less than about a 40%, less than about a 35%, less than about a 30%, less than about a 25%, less than about a 20% lung volume reduction, or less than about a 15% overall lung volume reduction. Such reduction may be achieved upon a single or multiple administrations of compositions of the present invention. A reduction of about 2% to about 3% overall lung volume reduction can be expected to produce a beneficial effect in a subject receiving such treatment, e.g., at least to a similar extent as that produced in LVRS.

Also in preferred embodiments, the coupling between targeting moieties is permanent or at least semi-permanent for a period of time between successive treatments as described herein, e.g., resisting biodegradation (e.g., hydrolysis) for the period of time between administrations of a composition of the present invention. In certain embodiments, at least about 70%, at least about 80%, at least about 90%, or at least about 98% of the coupling between targeting moieties remains intact for a period of time. In some preferred embodiments, the period is at least about one month, at least about 2 months, at least about 3 months, at least about 6 months, at least about a year, at least about 2 years, at least about 3 years, at least about 5 years, or at least about 10 years. In some preferred embodiments, the period is less than about 50 years, less than about 30 years, less than about 20 years, or less than about 15 years. In most preferred embodiments, the coupled targeting moieties keep some damaged lung tissue collapsed and/or sealed for the remainder of the life of the subject, for example, resisting biodegradation indefinitely. One of skill in the art will recognize that the permanence and/or biodegradability of the coupling between targeting moieties can depend on the coupling technique chosen and/or the coupling moiety used.

In preferred embodiments, some or all of the coupling moieties are strong enough to withstand mechanical pressures experienced within the lung. For example, the strain range corresponding to functional residual capacity during normal breathing does not result in breakage of at least about 30%, at least about 40%, at least about 50%, at least about 60%, at least about 70%, at least about 80%, at least about 90%, or at least about 95% of the coupling moieties in some preferred embodiments.

In some preferred embodiments, the coupling moieties exhibit a tear strength of at least about 50 g/sq. cm, at least about 100 g/sq. cm, at least about 200 g/sq. cm, or at least about 300 g/sq. cm. In some preferred embodiments, the coupling moieties exhibit a tear strength of less than about 5,000 g/sq. cm, less than about 3,000 g/sq. cm, less than about 1500 g/sq. cm, less than about 1300 g/sq. cm, less than about 1200 g/sq. cm, less than about 1000 g/sq. cm, less than about 800 g/sq. cm, less than about 600 g/sq. cm, or less than about 400 g/sq. cm.

Similarly, in preferred embodiments, the binding interaction between targeting moieties and their corresponding damage-correlated moieties is permanent or at least semi-permanent for a period of time between successive treatments as described herein, e.g., binding irreversibly, substantially irreversibly, or at least with a high binding constant to resist dissociation for the period of time between administrations of a composition of the present invention. For example, an alpha-1 antitrypsin moiety may form a pseudo-irreversible equimolar complex with neutrophil elastase in some embodiments. See, e.g., Sifers et al., “Genetic Control of Human Alpha-1 Antitrypsin”, Mol. Biol. Med., Vol. 6 pgs. 127-135 (1989). Without being limited to a particular theory or mode of action, the alpha-1 antitrypsin moiety may form an acyl-enzyme complex with its target. In some embodiments, binding can be further enhanced by genetic modification or by shuffling of known binding domains. As another example, a serpin moiety may react with its target protease to form a sodium dodecyl sulfate(SDS)-stable equimolar complex. Without being limited to a particular theory or mode of action, the complex between a serpin and its target protease may involve a covalent ester bond linkage, where an active site Serine residue of the protease binds a C-terminal residue of a cleaved form of the serpin to form an acyl-enzyme complex. See, e.g., U.S. Publication No. 2003/0216321. As yet another example, a monocyte elastase inhibitor moiety can form a covalent complex and/or an essentially irreversible complex with elastase. See, e.g., International Publication WO 96/10418 and U.S. Pat. No. 5,827,672.

In certain embodiments, at least about 70%, at least about 80%, at least about 90%, or at least about 98% of the targeting moieties remain bound to corresponding damage-correlated moieties for a period of time. In some preferred embodiments, the period is at least about one month, at least about 2 months, at least about 3 months, at least about 6 months, at least about a year, at least about 2 years, at least about 3 years, at least about 5 years, or at least about 10 years. In some preferred embodiments, the period is less than about 50 years, less than about 30 years, less than about 20 years, or less than about 15 years. In most preferred embodiments, the binding keeps some damaged lung tissue collapsed and/or sealed for the remainder of the life of the subject, for example, resisting dissociation indefinitely.

FIG. 1b illustrates one embodiment of a method to reduce lung volume using a composition comprising a first targeting moiety 104a and a second targeting moiety 104b wherein the targeting moieties are coupled. This figure provides an overview only, and is in no way intended to be limiting with respect to the present invention. For example, those skilled in the art will readily appreciate variations and modifications of the scheme illustrated. The figure schematically illustrates a terminal bronchiole 101 terminating in airspace of an alveolus 102. As mentioned above, the air space may be over-inflated and/or enlarged in certain pulmonary conditions, such as emphysema. Within the walls of the airspace, high amounts of damage-correlated moieties 103, 107 are found, for example, at different sites of damaged lung tissue and/or within the epithelial lining fluid.

A composition of the invention is administered, where the composition comprises a first targeting moiety 104a and a second targeting moiety 104b wherein the targeting moieties are coupled, for example, via a coupling moiety 108. FIG. 1b illustrates how, following administration, one of the targeting moieties 104a recognizes and binds its damaged-correlated moiety 107.

FIG. 1b also illustrates how the two targeting moieties can recognize and bind their corresponding damage-correlated moieties at two different sites within the air space. Following deflation, the walls of alveolus 102 are brought into closer proximity, allowing the second targeting moiety 104b to recognize and bind its damage-correlated moiety 103 at a different site of damaged lung tissue. The binding of coupled targeting moieties to hitherto further-apart damage-correlated moieties serves to help keep the walls of the air space closer together. A previously enlarged and/or distended alveolus may thus be kept in a collapsed and/or sealed state after re-inflation, thereby reducing lung volume.

Another aspect of the present invention provides a method of imaging damaged lung tissue by administering to a subject in need thereof a composition comprising an imaging moiety coupled to a targeting moiety that targets damaged lung tissue and imaging the damaged lung tissue. The targeting moiety acts to direct the administered composition to sites of damaged lung tissue, for example, by virtue of higher amounts of damage-correlated moieties in areas of the lung affected by a pulmonary condition compared with areas of the lung that are not affected or that are affected to a lesser extent. For example, where an alpha-1 antitrypsin moiety is used as a targeting moiety, the alpha-1 antitrypsin moiety can recognize and bind to elastase, which is found in higher concentrations at sites of damaged lung tissue in certain pulmonary conditions, e.g., in emphysema. The imaging moiety can then permit detection, preferably non-invasive and/or in vivo detection, of damaged regions. In emphysema, for example, regions of the lung with enlarged air spaces that have high amounts of elastase can be detected by the methods described herein.

Targeting imaging moieties to sites of damaged lung tissue can help reduce “background,” e.g., due to unbound imaging moieties at areas of the lung that are not affected by a pulmonary condition or that are affected to a lesser extent. In some preferred embodiments, e.g., unbound targeting moiety may be removed from the lungs, e.g., by deflating and/or collapsing, before detection of the imaging moieties.

In some embodiments, detection of the imaging moieties is carried out after allowing sufficient time for targeting of the administered compositions to areas of damaged lung tissue. In preferred embodiments, the targeting moiety recognizes and binds its damage-correlated moiety in at least about 30 minutes, at least about 20 minutes, at least about 10 minutes, or at least about minutes. In preferred embodiments, the targeting moiety recognizes and binds its damaged-correlated moiety in less than about 3 hours, less than about 2 hours, less than about 1 hour, less than about 45 minutes, less than about 30 minutes, less than about 15 minutes, or less than about minutes.

The imaging moiety may be imaged by any methods known in the art and/or described herein. For example, imaging may be carried out via traditional radiological techniques, including, for example the use of an X-ray, computer tomography (CT), and/or the use of more advanced techniques such as a positron emission tomography (PET) scan, nuclear scans, and/or scintigraphy, as well as magnetic resonance imaging (MRI), functional magnetic resonance imaging (FMRI), magnetoencephalography (MEG), and single photon emission computerized tomography (SPECT). Such imaging techniques can be used to detect bound imaging moieties in vitro or in vivo, preferably in vivo. High resolution scans, e.g., a high resolution CT scan, are preferable. In more preferred embodiments, such imaging produces a detailed map of the lungs, showing sites of damaged tissue and/or the extent of damage, e.g., by the relative amounts of imaging moieties bound to various sites within the lungs.

The method of detection used may depend on the imaging moiety administered. For example, ultrasound imaging can be used to detect an echogenic imaging moiety and/or an imaging moiety capable of generating an echogenic signal and/or other ultrasound imaging moieties. X-ray can be used to detect a heavy atom imaging moiety (e.g., having atomic weight of about 38 or above). Light imaging can be used to detect an imaging moiety capable of scattering and/or absorbing and/or emitting light. MR imaging can be used to detect an imaging moiety comprising a non-zero nuclear spin isotope (such as F-19) and/or an imaging moiety having unpaired electron spins. PET, scintigraphy, and/or SPECT can be used to detect a radionuclide imaging moiety.

For example, in some embodiments, an imaging moiety comprising a radioactive gamma emitter can be used, and can be detected via a gamma camera, scintillation counter, and/or other device capable of detecting gamma radiation. Radiation imaging cameras can use a conversion medium to absorb high-energy gamma rays and displace an electron, which emits a photon on its return a lower orbital state. Some cameras also use photoelectric detectors, e.g., arranged in a spatial detection chamber to determine the position of an emitted photon, as well as circuitry to analyze the photons detected in the chamber to help produce an image.

In embodiments using an imaging moiety comprising a magnetic species, e.g., a paramagnetic atom, the imaging moiety can be detected by MR imaging, e.g., a magnetic resonance imaging system can be used. In such systems, a strong magnetic field can be used to align nuclear spin vectors of atoms, such as paramagnetic atoms at sites of damaged lung tissue. The field can then be distributed by the paramagnetic atoms at such sites. As the nuclei return to equilibrium alignments, an image of sites of damaged lung tissue can be obtained.

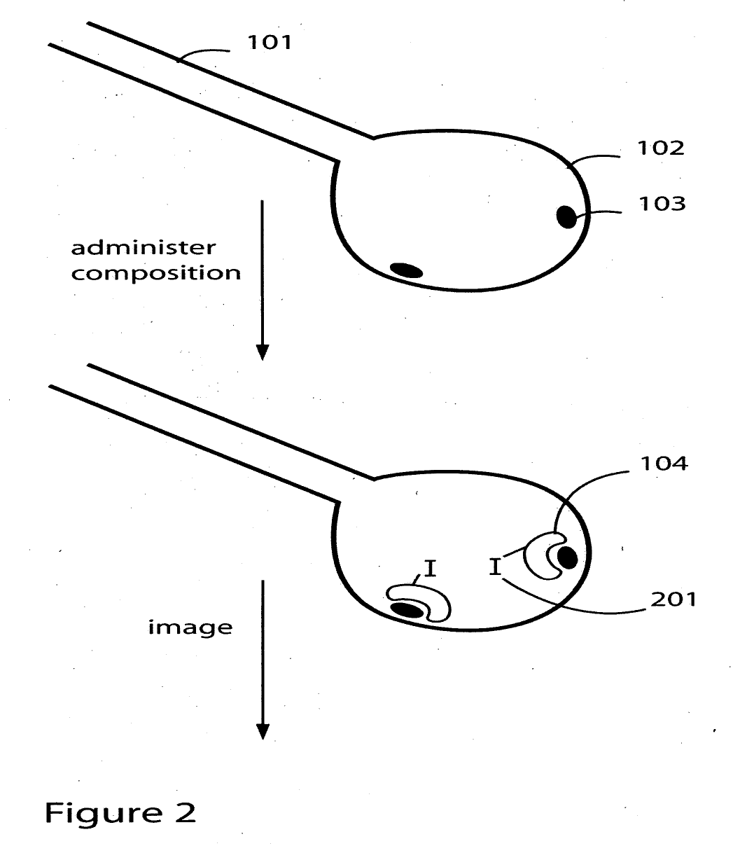

FIG. 2 illustrates one embodiment of a method to image damaged lung tissue using a composition comprising an imaging moiety coupled to a targeting moiety that targets damaged lung tissue. This figure provides an overview only, and is in no way intended to be limiting with respect to the present invention. For example, those skilled in the art will readily appreciate variations and modifications of the scheme illustrated. The figure schematically illustrates a terminal bronchiole 101 terminating in the airspace of an alveolus 102. As mentioned above, the air space may be over-inflated and/or enlarged in certain pulmonary conditions, such as emphysema. Within the walls of the airspace, high amounts of damage-correlated moieties 103 are found, for example, at sites of damaged lung tissue and/or within the epithelial lining fluid.

A composition of the invention is administered, where the composition comprises a targeting moiety 104 that targets damaged lung tissue, for example, by recognizing and binding its target damage-correlated moiety 103. In the illustrated embodiment, the composition also comprises an imaging moiety (I) 201. FIG. 2 illustrates how targeting moieties recognize and bind damage-correlated moieties at various sites of damaged lung tissue. Detection of the imaging moiety 201 by a suitable detection technique can provide an image of such damage, preferably facilitating diagnosis of a pulmonary condition.

Imaging methods described herein can afford detection of damaged lung tissue and preferably facilitate diagnosis and/or monitoring of the presence, extent, amelioration and/or worsening of a pulmonary condition, such as emphysema. Imaging methods described herein may be used in conjunction with treatment methods described herein, others currently known, and others to be developed. For example, detection of regions of damaged lung tissue may be followed by lung volume reduction surgery. Alternatively, the regions may indicate suitable positions for placement of a one-way valve and/or the need for sealing regions of damaged lung tissue, e.g., using compositions and/or methods described herein.

Some embodiments of the present invention employ both imaging and volume-reducing aspects of the invention described herein. For instance, damaged lung tissue may be imaged using a composition comprising an imaging moiety coupled to a targeting moiety. The extent of damage can indicate whether or not further treatment is needed and/or desirable. Such further treatment can include lung volume reduction by virtue of cross-linking compositions comprising a targeting moiety coupled to a cross-linkable moiety and/or by using a composition comprising coupled targeting moieties. In some embodiments, the imaging moiety may be coupled to a targeting moiety that itself is coupled to a cross-linkable moiety and/or one or more other targeting moieties. In some embodiments, a second composition comprising a targeting moiety coupled to a cross-linkable moiety and/or to one or more other targeting moieties can be used. In still some embodiments, lung volume reduction, e.g., using compositions and/or methods described herein, may be preceded and/or followed by imaging, e.g., and the images compared, e.g., to determine the extent of collapse and/or sealing achieved in regions of damaged lung tissue, preferably facilitating monitoring of the presence, position, extent and/or degradation of the cross-links and/or coupling moieties, and/or dissociation of the targeting moiety.

Administration of a composition comprising a targeting moiety, coupled to any or all of an imaging moiety, a cross-linkable moiety, a cross-linking activating moiety, other targeting moiety and/or other moiety and/or agent, may be followed by washing. The term “washing” as used herein refers to administration of a washing moiety that can facilitate removal of a targeting moiety from its respective damage-correlated moiety, e.g., making the damage-correlated moiety available for further binding to a subsequently-added targeting moiety. For instance, a washing step may follow administration and imaging of a composition comprising a targeting moiety coupled to an imaging moiety to free up target sites. Following washing, a composition comprising a targeting moiety coupled to a cross-linkable moiety and/or coupled to another targeting moiety may be administered to the subject, for example to achieve lung volume reduction by methods described herein. Washing moieties suitable for use in the present invention include, for example, soluble damage-correlated moieties that can compete with the damage-correlated moieties at sites of damaged lung tissue for binding with the targeting moieties. Preferably, the soluble damage-correlated moieties are modified so as to reduce and/or eliminate undesirable properties before administration to a subject. For example, a mutant elastase polypeptide may be used that can still bind to alpha-1 antitrypsin but that cannot degrade lung tissue or degrades lung tissue to a lesser extent than non-mutant elastase. Formulation, Routes of Administration, and Effective Doses

The targeting moieties, cross-linkable moieties, cross-linking activating moieties, and/or imaging moieties useful in the practice of the present invention can be delivered to a subject using a number of routes or modes of administration. The moieties may be delivered per se or as pharmaceutically acceptable salts thereof. The term “pharmaceutically acceptable salt” means those salts which retain the biological effectiveness, selected conformation and other desired properties of the moieties and/or agents of the present invention, and which are not biologically or otherwise undesirable. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the moiety contains a carboxyl group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

The targeting, cross-linkable, cross-linking activating, imaging moieties, and/or other moieties and/or agents or pharmaceutically acceptable salts thereof, can be formulated with a pharmaceutically acceptable carrier for administration to a subject in need thereof. “Pharmaceutically acceptable carriers” are well known in the pharmaceutical art, described, for example, in Remington's Pharmaceutical Sciences, Mack Publishing Co. (A. R. Gennaro edit. 1985). Suitable carriers include, for example, carriers like alcohol, DMSO, saline solution, and/or water. Pharmaceutical compositions for use in accordance with the present invention may be formulated in conventional manner using one or more physiologically acceptable carriers comprising excipients and/or auxiliaries, which facilitate processing of the active moieties into preparations that can be used pharmaceutically. Proper formulation is dependent upon the route of administration chosen.

In some embodiments, the compositions of the invention are dissolved in a suitable solvent, such as sterile water or PBS, and then dried to remove the solvent and produce a powder. Drying can be carried out in such as way as to retain the desired properties of the compositions, for example the capability of a targeting moiety to recognize and/or bind its target damage-correlated moiety. For example, vacuum concentration, spray drying, open drying, freeze-drying, and the like, can be used. The residue obtained can then be ground and/or further micronized.

In some preferred embodiments, the targeting, cross-linkable, cross-linking activating and/or imaging moieties, or pharmaceutically acceptable salt thereof, as well as other moieties and/or agents and/or pharmaceutically acceptable salts thereof, are formulated as dry powders or aerosolized physiologically acceptable solutions that may be delivered to the lungs of a subject. Power and/or liquid formulations can be prepared to facilitate administration, e.g., to facilitate transfer from the delivery device into the respiratory tract, preferably down to the alveoli distal to terminal bronchiles.

Powder formulations can be prepared in various ways, using conventional techniques. Powder formulations can be processed to improve ability to be delivered to a subject, e.g., via inhalation and/or trans-thoracically. For instance, the way in which the formulation flows through and/or out of an inhaler device or other device, can be improved by forming spherical agglomerates by, e.g., dry granulation processing. Spherical agglomerate can impart the compositions of this invention with superior handling characteristics. It is to be understood, however, that the present invention contemplates the use agglomerates and/or other particles of all shapes, including both spherical and non-spherical shapes. Power and/or liquid formulations also preferably have physical characteristics that help avoid clogging of an aerosol device and clumping of aerosolized material. For example, additives such as alcohol, soaps, surfactants, and/or Vitamin E may be use to help reduce clumping and to facilitate formation of small particles and/or droplets.

Liquid formulations may be produced by adding a volume of sterile delivery solvent to an amount of sterile composition of the present invention in powder or liquid form. In some embodiments, formulation temperatures of at least about 0° C., at least about 4° C., at least about 5° C., at least about 10° C., or at least about 15° C. may be used. In some embodiments, formulation temperatures of less than about 100° C., less than about 80° C., less than about 60° C., less than about 37° C., or less than about 30° C. may be used.

Formulation of the present invention may also be prepared to provide other suitable physiological parameters for use in the lungs, including for example, suitable pH. For instance, a pH of at least about 4, at least about 5, or at least about 6 may be used. In some embodiments, a pH of less than about 11.0, less than about 10.0, less than about 9.0, less than about 8, or less than about 7 may be used.

In preferred embodiments, formulation involves selecting parameters such as concentration, size and/or viscosity of targeting, cross-linkable, cross-linking activating and/or imaging moieties, as well as other moieties and/or agents, and/or pharmaceutically acceptable salts thereof, e.g., to provide a rheological profile, such that when aerosolized and/or nebulized, the formulation produces a range of particle and/or droplet sizes capable of being delivered to the lungs. A suitable mill, such as a jet mill, can be used to produce particles in a range of sizes that facilitates, or preferably maximizes, access to sites of damaged lung tissue, including sites distal to terminal bronchioles. In some embodiments, a nozzle comprising tapering pores may be used, e.g., to increase uniformity of the aerosol generated. See, e.g., U.S. Publication No. 2004/0124185.

In more preferred embodiments, a formulation is prepared that allows respiratory zone or deep lung delivery. In such embodiments, the formulation can yield a range of particle and/or droplet sizes adapted for delivery to the deep lung. In still more preferred embodiments, formulation involves selecting parameters such as concentration, size and/or viscosity of targeting, cross-linkable, cross-linking activating and/or imaging moieties, as well as other moieties and/or agents, and/or pharmaceutically acceptable salts thereof, such that when aerosolized and/or nebulized, the formulation produces a range of particle and/or droplet sizes capable of being delivered to the lung alveoli, preferably to a lung alveolus distal to a terminal bronchiole.

Droplets and/or particles of suitable size ranges can be obtained by selecting appropriate delivery devices, molecular weight, concentration, and/or additives as known in the art and/or described herein. See, e.g., U.S. Publication No. 2002/0086842. For example, various formulations can be screened to determine ones that produce droplet and/or particle size in desired ranges.

In preferred embodiments, the compositions of the present invention are administered via the respiratory tract, e.g., via inhalation. The term “inhalation” includes inhalation via the mouth, nose, tracheae, or any combination thereof. A pharmaceutical formulation for administration via inhalation may be made up according to techniques known in the pharmaceutical arts and administered via aerosol inhalation, dry powder inhalation, liquid inhalation, and/or instillation. For example, a diagnostically and/or therapeutically effective amount of a composition of the invention may be delivered by inhalation of a breathable mist by the animal subject.

Preparation of inhalable formulations are known in the art, e.g., see U.S. Publication No. 2003/0232019 and International Publication No. WO 2004/054556. For example, a composition of the present invention can be formulated with a breathable fluorocarbon propellant. Inhalable preparations preferably provide droplets and/or particles with median mass distribution size of at least about 0.1 microns, at least about 0.3 microns, at least about 0.5 microns, at least about 1 micron, or at least about 2 microns. Inhalable preparations preferably provide droplets and/or particles with median mass distribution size of less than about 20 microns, less than about 15 microns, less than about 10 microns, less than about 6 microns, less than about 5 microns, less than about 3 microns, or less than about 2 microns. Particle and/or droplet sizes are preferably between about 2 microns to about 5 microns.

Size may be selected to allow compositions of the present invention access to sites of damaged tissue in various lung regions. The respiratory system can be divided into three regions: (i) the tracheal/pharyngeal region, (ii) the bronchial region, and (iii) the alveolar region. Droplets and/or particles of about 10 microns to about 50 microns typically migrate to the tracheal/pharyngeal and/or bronchial region of the lungs; while droplets and/or particles of about 0.5 microns to about 5 microns, e.g., droplets and/or particles of about 2 microns, typically migrate to the alveolar region. Larger sizes may not as efficiently reach alveoli through distal bronchioles. Smaller droplets and/or particles may be exhaled by the subject before the targeting moiety contacts its damage-correlated moiety. Droplet and/or particle size of compositions of the present invention can be measured by techniques known in the art, including, e.g., those described herein.

Various physical parameters may be used to facilitate access of compositions of the present invention to various sites of damaged tissue within the lungs. For example, the mass median aerodynamic diameter (MMAD), usually expressed in microns, can be used to predict where a droplet and/or particle distributes in the lungs. Mass Median Aerodynamic Diameter can be measured using a Cascade Impactor relating to size of compositions of the present invention. A humidified Cascade Impactor is preferably used to better reflect conditions of pulmonary delivery. Further, particle size distribution can also be measured with a Malvern Laser, for example. The geometric standard deviation (GSD) is another parameter that can be used. A GSD of about 1 correlates to a normal distribution. A GSD of less than about one can indicate a narrow size dispersion while a GSD of more than about 1 can indicate a broad size dispersion. Such parameters are further influenced by the ability of a targeting moiety of a composition of the present invention to recognize and/or bind to its target damage-correlated moiety.

Charge may also be used to facilitate aerosol formation. For example, in some embodiments, droplets and/or particles can be made to carry a negative charge. The like charges can repel each other, helping to disperse the particles and/or droplets into an aerosol cloud by, e.g., by electrostatic forces. Like positive charges on particles and/or droplets may also be used in a similar manner.

Animal models can also be used to determine suitable ranges of droplet and/or particle size for delivery of compositions of the present invention to damaged lung tissue, e.g., see Raabe et al., Ann. Occup. Hyg., Vol. 32 pgs. 53-63 (1998) (surveying access of particle size to various regions of the lungs in laboratory animals).

Solution or liquid formulations may be aerosolized to form a breathable mist via, e.g., a device such as an inhaler, a nebulizer, and/or an atomizer. In some embodiments, the formulation is a dry power, which can be made up into solution, e.g., with saline or water before aerosolization. In still some embodiments, a dry powder can be delivered per se by a device such as an intra alveolar device (IAD), an air gun powered aerosol chamber, and/or other dry powder delivery devices, e.g., from Dura Delivery Systems and/or Glaxo Wellcome.

A composition of the present invention may be aerosolized by any techniques known in the art, described herein, and/or that can be developed. For example, the composition may be pressurized through micro pores and then blown through an inline blower, such as a high-pressure fan system. The fan or pump is preferably timed to coincide with the time of inspiration or a time just before inspiration. In some embodiments, for example, the delivery of the compositions can be metered as a function of the in-flow volume.

The aerosolized composition can be delivered by any methods known in the art and/or described herein. For example, the composition can be infused under pressure directly into a bronchus and/or into an enlarged air space. In some embodiments, a catheter can be used to suck air out of a less distal lumen of the lungs through another path. In some embodiments, the composition can be infused into an enlarged air space using a first catheter while sucking air out with a second catheter through another path leading from the same air space, e.g., from another bronchi branch, to get a circular flow path. In yet another approach, the flow around a catheter or other infusion device can be blocked using balloons, covered braid structures, expanding foam, flaps that make one-way valves, and/or expanding corrugations.

Compositions of the present invention may also be administered via inhalation using a portable (e.g., hand held) inhaler device, such as devices used to deliver anti-asthmatic agents or anti-inflammatory agents. For example, a fine dry powder can be delivered as an aerosol by compressing air into the powder inside the inhaler. This can disperse the powder as a cloud of particles, preferably of the size ranges that allow access to alveoli distal to terminal bronchioles.

In some embodiments, the inhaler device may be designed to deliver single or multiple doses, minimizing risks from accidental large doses, and protecting the formulation from light, excessive moisture, and/or other contaminants. Dry powder and metered dose inhalers can be used to administer compositions of the invention to the pulmonary air passages of a subject in need thereof. Metered dose inhalers can deliver medicaments in a dispersion and/or in solubilized form. These inhalers can include a relatively high vapor pressure propellant, which forces aerosolized material into the respiratory tract upon activation of the device.

Some embodiments involve delivery by nebulization to the lungs, where, e.g., the delivery device can be a nebulizer. For example, a nebulizer can be used that generates an aerosol containing the compositions of the present invention, preferably an aerosol of droplets and/or particles of less than about 10 microns. Nebulizers are known in the art, and include, e.g., a jet nebulizer, which can be an air or liquid jet nebulizer; an ultrasonic nebulizer; a compressed air nebulizer (e.g., an AeroEclipse, Pari L. C., a Parijet; and/or a Whisper Jet) and/or a pressure mesh nebulizer. Compressed air nebulizers can generate droplets by using fast moving air to shatter a liquid stream. Ultrasonic nebulizers can nebulize a liquid solution using ultrasonic waves, e.g., by using a piezoelectric transducer to transform electrical current into mechanical oscillations; while pressure mesh nebulizers force fluid through a mesh-like surface under pressure. The nebulizer may use a pressure of at least about 5 psi, at least about 10 psi, at least about 15 psi, at least about 20 psi, at least about 25 psi, or at least about 30 psi. The nebulizer may use a pressure of less than about 60 psi, less than about 50 psi, or less than about 40 psi. For administration using a nebulizer, a subject can inhale aerosolized composition of the present invention via continuous neblulization, e.g., in a manner similar to that used to administer aerosolized bronchodilators. For example, the aerosol may be delivered via tubing or a mask to the mouth and/or nose, as well as by using an Ambu bag, blow-by mask, endotracheal tube, nasal cannula, nasal covering, and/or nonrebreather.

A suitable volumetric flow rate (L/min) for the nebulizer may be selected. It is preferable that the volumetric flow rate not exceed twice the subject's minute ventilation, as the average inspiratory rate is about twice the minute ventilation with exhalation and inhalation each representing about half of the breathing cycle. For example, a nebulizer with a volumetric flow rate of less than about 20 L/min, less than about 15 L/min or less than about 10 L/min may be used. A nebulizer can also be selected to generate desired ranges of particle and/or droplet size. Along with volumetric flow rate, various factors may be considered as will be appreciated by one of skill in the art. Such factors include aerosol mass output (mg/L) and/or retained volume (mL). For example, with respect to a compressed air nebulizers, factors such as air flow, hole diameter, and/or air pressure can influence size distribution. With respect to an ultrasonic nebulizer, factors include rate of air flow, hole diameter, and/or ultrasound frequency,

Administration can also involve delivery of aerosolized droplets and/or powders of the present invention under positive pressure ventilation. For example, a device such as a Continuous Positive Airway Pressure device can be used to afford ventilatory assistance. This assistance can facilitate access of the compositions of the present invention to sites of damaged tissue in alveoli of the deep airways. Additionally, positive end expiratory pressure may be used to provide further assistance in this regard. In some embodiments, a device can be used that delivers a composition of the present invention when the subject produces a level of negative inspiratory pressure, e.g., at inspiratory flow rates.

Other devices that may be used include, for example, include a canister adapted to contain a preparation comprising a composition of the present invention under pressure. The canister may feature a valve, e.g., for regulating delivery of the preparation; a nozzle connected to the valve for converting the pressurized preparation inside the canister into an inhalable aerosol mist upon actuating the valve. See, e.g., U.S. Publication No. 2002/0086852. Other devices for delivery of compositions of the present invention to the lungs of a subject in need thereof include a spray atomizer.

Compositions of the present invention can also be delivered in a non-aerosolized form. Further, any combination of aerosol and/or non-aerosol forms may be used.

For example, a liquid, solution, suspension, viscous liquid, liquid film, slurry, foam, and/or thicksotropiec form(s) may be used. Any of such forms can be delivered to the lungs by any techniques known in the art, to be developed, and/or described herein. For example, a liquid, solution, suspension, viscous liquid, liquid film, slurry, foam, and/or thicksotropiec form can be administered by fluid washings, liquid ventilation, bolus liquid drip, and/or pulmonary lavage. In some embodiments, a fluorochemical medium may be used.

Administered solutions may include, for example, physiologically acceptable solutions of targeting, cross-linkable, cross-linking activating and/or imaging moieties (and/or other moiety and/or agents) of the present invention. After delivery to the lungs or a first portion thereof, the solvent can evaporate and/or dissipate such that the targeting moiety, cross-linkable moiety, cross-linking activating and/or imaging moiety (and/or other moiety and/or agent) is left behind.

In still some embodiments, the compositions may be delivered as solids, semi-solids, solid films, hydrogels, agars, and/or sol-gels. For example, compositions of the present invention may be administered as an absorbable sponge, e.g., as an absorbable gelatin sponge (e.g., GelfoaMTM) and/or as an absorbable wax. Non-absorbable waxes may also be used. Further, in some embodiments, petroleum-based compounds (e.g., petrolatum), latex, natural or synthetic rubber, starches, and/or alginate compounds may be used in formulating compositions of the present invention.

In some embodiments, compositions of the present invention are delivered to the lungs via instillation, e.g., direct instillation through the trachea, e.g., through the anterior aspect of the trachea. The compositions of the present invention can be administered as a liquid solution, including, e.g., an aqueous solution comprising water or a buffered physiological solution, such as saline. Instillation administration can be carried out over a period of at least about 2 minutes, at least about 5 minutes, at or least about 10 minutes. The instillation period may be less than about 30 minutes, less than about 20 minutes, or less than about 15 minutes. The length of instillation time may be selected based on a number of factors, including the composition used, the extent of the damage, and the like. Instillation may involve delivery via bronchoscopy and/or endoscopy.

Other techniques for delivering compositions of the present invention to damaged lung tissue may also be used, including, e.g., use of an impregnated applicator tip, e.g., U.S. Pat. No. 5,928,611; and/or an applicator for delivering liquid and/or semi-liquid compositions via laproscopy and/or endoscopy, e.g., U.S. Pat. No. 6,494,896. Fibers, micro fibers, lattice-work stents, filagree designs, and/or porous structures may also be used, e.g., where the structure is coated with a composition of the present invention.

The compositions of the present invention can also be delivered via trans-thoracic administration. For example, in some embodiments, air spaces can be targeted directly through the ribs for more controlled localization, e.g., being applied through a scope. Trans-thoracic delivery may involve delivery into the pleural space using a needle percutaneously, and/or using a catheter and/or chest tube. In some embodiments, compositions can be delivered via bronchoscopy and/or use of an endotracheal tube. Such embodiments, however, are less preferred as discussed above. Compositions of the present invention can also be delivered to the lungs during liquid ventilation or pulmonary lavage using a fluorochemical medium.

The compositions of the present invention can also be given intravenously. For example, the pharmaceutical and/or diagnostic compositions of the present invention may be formulated with a pharmaceutically acceptable carrier to provide sterile solutions or suspensions for administration via injection. Injectables can be prepared in conventional forms, e.g., as liquid solutions, suspensions, and/or solid forms suitable for making a solution or suspension in liquid prior to injection, and/or as emulsions. Suitable excipients that may be used include, for example, water, saline, dextrose, mannitol, lactose, lecithin, albumin, sodium glutamate, cysteine hydrochloride, and the like. In some embodiments, pharmaceutical compositions for injection may contain auxiliary substances, such as wetting agents, pH buffering agents, and the like. For example, a carbonate/bicarbonate buffer system may be used.

In some embodiments, the compositions of the invention are administered using a delivery vehicle. A “delivery vehicle” as used herein refers to any particle that can be used to carry compositions of the present invention. Examples of delivery vehicles include, but are not limited to, liposomes, viral, bacteriophage, cosmid, plasmid, and fungal vectors and other recombinant vehicles typically used in the art.

Delivery vehicles can carry a composition of the present invention encoded by a polynucleotide sequence. Expression of the sequence can produce the composition e.g. a fusion polypeptide of two or more coupled targeting moieties.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to enhance in vitro transcription and/or expression, it may be necessary to remove, add, and/or alter 5′ and/or 3′ untranslated portions to eliminate extra, potentially inappropriate alternative translation initiation codons, or other sequences that may interfere with or reduce expression, either at the level of transcription or translation. In some embodiments, consensus ribosome binding sites can be inserted immediately 5′ of the start codon to enhance expression.

In some embodiments, a viral vector can be used. A viral vector can include a natural or recombinantly produced virus or viral particle that comprises a polynucleotide to be delivered, either in vivo, ex vivo or in vitro. Examples of viral vectors include baculovirus vectors, retroviral vectors, adenovirus vectors, adeno-associated virus vectors and the like. A viral vector can enter a host cell via its normal mechanism of infection or can be modified such that it binds to a different host cell, e.g., by binding to a different surface receptor or ligand to enter the different host cell.

Delivery vehicles can also include non-viral vectors, including liposome complexes. Liposomes may comprise an aqueous concentric layer adherent to a hydrophopic or lipidic layer. The hydrophobic layer may comprise, for example, phospholipids, such as lecithin and sphingomyelin, steroids such as cholesterol, as well as ionic surface active substances such as dicetyphosphate, phosphatidic acid, stearylamine, and the like. Various liposome complexes known in the art may be used to aid delivery of the compositions of the present invention to the lungs, in aerosol and/or non-aerosol formulation. For example, particulate formulations combining compounds having biocompatible hydrophobic domains with conjugates having both hydrophobic and hydrophilic regions may be used. See, e.g., U.S. Pat. No. 6,500,461. In some embodiments lipid vesicles may be used comprising bilayers with a salt form of an organic acid derivative of a sterol, as described, e.g., in U.S. Pat. No. 6,352,716. In some embodiments, the use of liposome complexes can facilitate delivery of compositions of the present invention, e.g., by keeping the composition intact and/or in appropriate conformation necessary and/or involved in the recognition and/or binding to a damage-correlated moiety.

In still some embodiments, liposomes containing compositions of the invention are coated with, e.g., a hydrophilic agent, such as hydrophilic polymer chains like polyethylene glycol (PEG). Examples of PEG-liposomes are known in the art, e.g., see U.S. Publication No. 2003/0138481 and U.S. Publication No. 2003/0113369. In some embodiments, the targeting moiety may be coupled to exposed PEG chains to facilitate targeting of its damage-correlated moiety. In some embodiments, the hydrophilic chains may temporarily shield the targeting moiety from interaction with its target damage-correlated moiety. Such liposomes are described, e.g., in U.S. Publication No. 2004/0009217.

In some embodiments, liposome complexes may facilitate targeted delivery to areas of damaged lung tissue. For instance, peptide-lipid conjugates may be incorporated into liposomes, for example, to selectively destabilize the liposomes in the vicinity of damage-correlated moieties, e.g., in the vicinity of higher concentrations of elastase or other damage-correlated moieties in areas affected by a pulmonary condition compared with areas of the lung that are not affected or that are affected to a lesser extent. See, e.g., peptide-lipid conjugates described in US. Pat. No. 6,087,325.

Delivery vehicles can also include other delivery systems associated with membranes (e.g., biocompatible or bioerodable membranes), including, e.g., dendrimer-based methods and compositions for targeting delivery. See, e.g., US Publication No. 2004/0120979. See also, e.g., US Publication No. U.S. 2003/0064050, describing dendritic polymer conjugates useful as drug delivery systems. For example, a dentritic polymer conjugate useful as a delivery system in the practice of the present invention can comprise a dendritic polymer coupled to a targeting moiety described herein.

In some embodiments, a composition of the present invention may be used with a moiety that increases solubility and/or pharmacologic compatibility of the targeting, cross-linkable, cross-linking activating and/or imaging moiety, as well as other moieties and/or agents, for example, by enhancing hydrophobicity. For example, in some embodiments, absorption enhancing preparations (e.g., liposomes described above) may be utilized. Moieties that may be co-administered to achieve such effects include, for example, amphotericin B, betamethasone valerete, beclomethasone, cortisone, dexamethasone, DPPC/DPPG phospholipids, doxorubicin, estradiol, isosorbide dinitrate, nitroglycerin, prostaglandins, progesterone, testosterone, and/or vitamin E, and/or esters of any of these.

Compositions for use in treating and/or detecting pulmonary conditions preferably have low levels of toxicity during useable life and are preferably sterilized. Sterilization may be accomplished by techniques known to in the art, including, for example, chemical, physical, and/or irradiation methods. Physical methods can include sterile fill, filtration, use of heat (dry or moist) and/or retort canning. Irradiation methods of sterilization can include gamma irradiation, electron beam irradiation, and/or microwave irradiation. Preferred methods are dry and moist heat sterilization and electron beam irradiation. Different moieties of the invention can be sterilized separately, e.g., as described in EP 1433486, e.g., to form final sterile compositions.

Preferably, the compositions of the present invention have a bacterial count of less than about 2 cfu/g, less than about 1 cfu/g, or less than about 0.1 cfu/g. Such precautions can reduce abscess formation. Preservatives may also be used including, but not limited to, hydroquinone, pyrocatechol, resorcinol, 4-n-hexyl resoreinol, captan (i.e., 3 α,4,7,7 α-tetrahydro-2-((trichloromethyl)thio)-1H-isoindole-1,3 (2H)-dione), benzalkonium chloride, benzalkonium chloride solution, benzethonium chloride, benzoic acid, benzyl alcohol, cetylpyridinium chloride, chlorobutanol, dehydroacetic acid, o-phenylphenol, phenol, phenylethyl alcohol, potassium benzoate, potassium sorbate, sodium benzoate, sodium dehydroacetate, sodium propionate, sorbic acid, thimerosal, thymol, phenylmercuric compounds such as phenylmercuric borate, phenylmercurie nitrate and phenylmercuric acetate, formaldehyde, and formaldehyde generators such as the preservatives Germall II.RTM. and Germall 115.TM. (imidazolidinyl urea, available from Sutton Laboratories, Charthan, N.J.) and the like. Further, preferred preparations contain nontoxic concentrations of toxins, such a heavy metals, for example, using established criteria for USP water for inhalation.

The present invention also encompasses diagnostic and/or pharmaceutical compositions prepared for storage before administration. Such compositions preferably contain preservatives and/or stabilizers. For example, sorbic acid and/or esters of phydroxybenzoic acid may be added. In addition, antioxidants and suspending agents may be used.

Pharmaceutical and/or diagnostic compositions useful in this invention may also include stabilizing agents, e.g., to reduce premature cross-linking. Stabilizing agents can include, e.g., vapor phase stabilizers, such as an anionic vapor phase stabilizer, and/or liquid phase stabilizers, e.g., an anionic liquid phase stabilizer. Such stabilizing agents may also include radical stabilizing agents, and/or a mixture of various stabilizing agents, preferably where the mixture does not interfere with, retard, and/or prevent the desired reaction. See, e.g., U.S. application Ser. No. 09/099,457.

If necessary or desirable, the compositions of the present invention may be administered in combination with one or more other therapeutic agents. The choice of therapeutic agent that can be co-administered with a composition of the present invention will depend, in part, on the condition being treated and the desired effect to be achieved. Coupling of such agents to a targeting moiety of the present invention can, e.g., improve efficacy, for example by targeting the drug to sites of damaged lung tissue.

For example, the composition may be administered with a growth factor, an anti-surfactant and/or an antibiotic or other therapeutic agent, including small molecule or polypeptide drugs. Examples of growth factors that may be used include a fibroblast growth factor, a transforming growth factor-β₁, and/or a platelet-derived growth factor (PDGF), as well as functional analogs thereof. Determination of dosage ranges are well within the knowledge and/or skill of those in the art, e.g., about 1 to about 100 nM of polypeptide growth factor can be used.

Examples of antibiotics that may be used include ampicillin, sisomicin, cefotaxim, gentamycin, penicillin, nebacetin, and the like. Additionally, in some embodiments, antimicrobial agents, antiviral agents, antiseptics, bacteriocins, disinfectants, anesthetics, fungicides, anti-inflammatory agents, or other active agents or mixtures thereof may be administered with a composition of the present invention. Such compounds can include acetic acid, aluminum acetate, bacitracin, bacitracin zinc, benzalkonium chloride, benzethonium chloride, betadine, captan (i.e., 3 α,4,7,7 α-tetrahydro-2-((trichloromethyl)thio)-1H-isoindole-1,3 (2H)-dione), benzalkonium chloride, benzalkonium chloride solution, benzethonium chloride, benzoic acid, benzyl alcohol, bleomycin, calcium chloroplatinate, cephalosporin, certrimide, cetylpyridinium chloride, chlorobutanol, cloramine T, chlorhexidine phosphanilate, chlorhexidine, chlorhexidine sulfate, chloropenidine, chloroplatinatic acid, ciprofloxacin, clindamycin, clioquinol, cresol, chlorocresol, cysostaphin, dehydroacetic acid, doxorubicin, formaldehyde, gentamycin, hydroquinone, hydrogen peroxide, iodinated polyvinylidone, iodine, iodophor, imidazolidinyl urea, minocycline, mupirocin, neomycin, neomycin sulfate, nitrofurazone, non-onynol 9, o-phenylphenol, phenylmercuric additives such as phenylmercuric borate, phenylmercurie nitrate and/or phenylmercuric acetate phenol, phenylethyl alcohol, potassium benzoate, potassium sorbate, potassium permanganate, polymycin, polymycin B, polymyxin, polymyxin B sulfate, polyvinylpyrrolidone iodine, povidone iodine, 8-hydroxyquinoline, preservatives (e.g., alkyl parabens and salts thereof, such as butylparaben, ethylparaben, methylparaben, methylparaben sodium, propylparaben, propylparaben sodium, and/or pyrocatechol), quinolone thioureas, rifampin, rifamycin, resorcinol, 4-n-hexyl resoreinol, silver acetate, silver benzoate, silver carbonate, silver chloride, silver citrate, silver iodide, silver nitrate, silver oxide, silver sulfate, sodium benzoate, sodium dehydroacetate, sodium propionate, sorbic acid, sodium chloroplatinate, sodium hypochlorite, sphingolipids, sulfonamide, tetracycline, sulfadiazine salts (such as silver, sodium, and zinc), thimerosal, thymol, tiotropium bromide, zinc oxide, and the like, and any combinations thereof.

Other drug moieties that may be co-administered include, for example anti-oxidants, atropine methyl nitrate, albuterol (salbutamol) sulfate, alcetylcysteine, anticholinergics, atriopeptin, bitolterol mesylate, beta agonists, other bronchodilators, e.g., isoetharine, methylxanthines, captopril, calcitonin, cromolyn sodium, cyclosporin, ephedrine sulfate, ephedrine bitartrate, epidermal growth factor, etoposide, fluroisolide, heparin, ibuprofin, insulin, interferon, isoetharine hydrochloride, insulin, interleukin-2, isoetharine mesylate, isoproteranol hydrochloride, isoproteranol sulfate, leukotriene inhibitors, lipase inhibitors, lipocortin, lung surfactant protein, mast cell stabilizers, metaproteranol sulfate, narcotics, n-acetyl cysteine, pentamidin, non-steroidal anti-inflammatory drugs (NSAIDs), peptides, phosphodiesterase inhibitors, phospholipase inhibitors, plasma factor 8, procaterol, propranalol, pulmozyme (Genentech), P2Y2 receptor agonists, steroids, superoxide dismutase, terbutaline, terbutaline sulfate, theophylline, tissue plasminogen activator (TPA), tobermycin, tumor necrosis factor, vasopressin, and/or verapamil.

Further, the composition may also be administered with a nucleic acid, e.g., a nucleic acid encoding a polypeptide, antisense oligonucleotide, or interfering RNA (e.g., siRNA). Compositions of the present invention may also serve as “depot” for slow release of therapeutic moieties or other active agents at sites of damaged lung tissue.

All formulations for aerosol, trans-thoracic, instillation, intravenous and/or other administration can be formulated in dosages suitable for administration. Diagnostic and/or pharmaceutical compositions suitable for use in the present invention include compositions wherein the moieties are present in an effective amount, i.e., in a diagnostically and/or pharmaceutically effective amount. A diagnostically effective amount includes a sufficient amount of a composition comprising an imaging moiety to allow detection of the presence of the imaging moiety, preferably at a site of damaged lung tissue, and more preferably by a non-invasive and/or in vivo imaging technique. A pharmaceutically effective amount includes a sufficient amount of a composition comprising a targeting moiety, cross-linkable moiety, cross-linking activating moiety (and/or other moiety and/or agent) to produce a therapeutic and/or a prophylactic benefit in at least one pulmonary condition being treated. The effective amount can be administered in a single dose or in a series of doses separated by appropriate time intervals, such as minutes, hours, or days. The actual amount effective for a particular application will depend on the pulmonary condition being detected and/or treated, the route of administration used, the identity of the targeting, cross-linkable, cross-linking activating, imaging moieties and/or other moieties and/or agents to be used, and other consideration that will be appreciated by those of skill in the art. Determination of an effective amount is well within the capabilities of those skilled in the art, especially in light of the disclosures herein.

The effective amount when referring to a composition comprising a targeting, cross-linkable, cross-linking activating, imaging moiety, and/or other moiety and/or agent will generally mean the dose ranges, modes of administration, formulations, etc., that have been recommended or approved by any of the various regulatory or advisory organizations in the medical or pharmaceutical arts (e.g., FDA, AMA) or by the manufacturer or supplier. The effective amount when referring to producing a benefit in treating a pulmonary condition, such as emphysema, will generally mean the amount that achieves clinical lung volume reduction recommended or approved by any of the various regulatory or advisory organizations in the medical or surgical arts (e.g., FDA, AMA) or by the manufacturer or supplier.

A person of ordinary skill using techniques known in the art can determine the effective amount of the targeting moiety, cross-linkable moiety, cross-linking activating moiety, imaging moiety, and/or other moiety and/or agent of the composition to be administered. The effective amount may depend on the moiety and/or agent to be used, and can be deduced from known data, e.g., data regarding binding constants for a targeting moiety, concentrations to achieve cross-linking for cross-linkable and cross-linking activating moieties, and sufficient imaging moiety to permit detection.

In some embodiments, dosages can be at least about 0.001 μg/kg/body weight, at least about 0.005 μg/kg/body weight, at least about 0.01 μg/kg/body weight, at least about 0.05 μg/kg/body weight, or at least about 0.1 μg/kg/body weight. In some embodiment, dosages can be less than about 0.05 mg/kg/body weight, less than about 0.1 mg/kg/body weight, less than about 0.5 mg/kg/body weight, less than about 1 mg/kg/body weight, less than about 2 mg/kg/body weight, less than about 3 mg/kg/body weight, or less than about 5 mg/kg/body weight of a composition of the invention. In some embodiment, dosages can be less than about 10 mg/kg/body weight, less than about 25 mg/kg/body weight, less than about 50 mg/kg/body weight, less than about 75 mg/kg/body weight, less than about 100 mg/kg/body weight, less than about 150 mg/kg/body weight, or less than about 200 mg/kg/body weight of a composition of the present invention.

The dosage may vary depending on the moieties used and their known biological properties. For example, it is known that fibrinogen comprises about 2 to about 4 g/L blood plasma protein and is cleaved to fibrin upon exposure to thrombin at the initiation the blood clotting cascade. In the context of reducing lung volume, formulations can be prepared containing useful concentrations of fribnogen and/or fibrin as a cross-linkable moiety and thrombin, batroxobin, a thrombin receptor agonist, and/or calcium as a cross-linking activating moiety. For example, a formulation comprising at least about 1%, at least about 2%, at least about 3%, at least about 4%, at least about 5%, at least about 8%, at least about 10%, at least about 12%, or at least about 15% fibrinogen may be used (e.g., in saline solution, for instance about 0.8%, about 0.9%, about 1%, or about 1.2% saline), and may be activated using at least about 0.5, at least about 1, at least about 5, at least about 10, or at least about 12 units of thrombin per ng of fibrinogen, and/or more than about 1 mM, more than about 1.5 mM, more than about 3 mM, more than about 5 mM, or more than about 8 mM calcium (e.g., in a CaCl₂ solution). Some embodiments may use a preparation of less than about 40 mM, less than about 30 mM, or less than about 20 mM calcium (e.g., in a CaCl₂ solution). Additionally, at least about 0.5%, at least about 1%, at least about 3%, at least about 5%, or at least about 6% of factor XIIa transglutaminanse may also be used to promote cross-linking. Formulation of fibrin-based compositions for achieving cross-linking are also known in the art, e.g., and may contain about more than about 10 mg/ml, more than about 20 mg/ml, more than about 25 mg/ml, or more than about 50 mg/ml. Fibrin-based compositions useful in the practice of this invention may also contain less than about 250 mg/ml, less than about 200 mg/ml, less than about 150 mg/ml, less than about 100 mg/ml, or less than about 50 mg/ml. See, e.g., other fibrin sealant compositions as provided in e.g., U.S. Pat. No. 5,739,288.

Further, the effective amount for use in humans can be determined from animal models, e.g., mice, rabbits, dogs, sheep, or pigs. For example, emphysema can be induced in C57BL/6 mice by administering nebulized porcine pancreatic elastase (about 30 IU/day for about 6 days), as described, for instance, in Ingenito et al., Tissue heterogeneity in the mouse lung: effects of elastase treatment, Articles in Press. J Appl Physiol (Mar. 12, 2004). 10.1152/japplphysiol.01246.2003. Similarly, emphysema-like conditions may be induced in sheep exposed to papain (inhalation of about 7,000 units/week for four consecutive weeks). Emphysema can also be induced in animal models by exposure to cadmium chloride, high concentrations of oxygen, and/or cigarette smoke. Ingenito, et al., “Bronchoscopic lung volume reduction using tissue engineering principles”, American Journal of Respiratory and Critical Care Medicine, Vol. 167 pgs. 771-778 (2003). A dose suitable for sealing damaged lung tissue in humans can be formulated based on doses found to be effective in animal models in reducing lung volume and freeing up space for expansion of remaining non-damaged or healthier tissue. Other techniques would be apparent to one of ordinary skill in the art. Further, the amount of administered composition comprising a cross-linkable moiety and/or coupled targeting moieties can be selected to be not so large as to generate high local hydrostatic pressures, preferably avoiding local hydrostatic pressures that exceed capillary perfusion pressure that can lead to abscess formation.

Similarly, a dose for imaging damaged lung tissue in humans can be formulated based on that used to image in the lungs of a suitable animal model. Diagnostic compositions comprising a targeting moiety and an imaging moiety can be prepared using a pharmaceutically acceptable carrier and a diagnostically effective amount of the composition. Diagnostically effective amount required as a dose to allow imaging will depend upon the route of administration, the condition being treated, the targeting moiety being used, the imaging moiety being used, and the diagnostic detail sought to be obtained, as well as other factors that will be appreciated by those of skill in the art of medical diagnostics. One of skill in the art of medical diagnostics will readily be able to determine suitable dosages, especially in light of the disclosures provided herein.

In preferred embodiments, the dose for imaging is sufficient to detect the presence of an imaging moiety at a site of damaged lung tissue. For example, in some embodiments, radiological imaging can require that the dose provide at least about 3 μC, at least about 5 μC, or at least about 10 μC of imaging moiety. In some embodiments, radiological imaging can require that the dose provide less than about 30 μC, less than about 20 μC, or less than about 15° C. of imaging moiety. Some embodiments using magnetic resonance imaging can require a dose of at least about 0.0005 mmol/kg, at least about 0.001 mmol/kg, at least about 0.005 mmol/kg, at least about 0.01 mmol/kg, at least about 0.05 mmol/kg, at least about 0.1 mmol/kg, at least about 0.5 mmol/kg, or at least about 1 mmol/kg of imaging moiety to body weight of the subject. In some embodiments, magnetic imaging can require a dose of less than about 10 mmol/kg, less than about 8 mmol/kg, less than about 5 mmol/kg, less than about 3 mmol/kg, or less than about 2 mmol/kg of an imaging moiety to the body weight of the subject. As a further example, iodine may be used as an imaging moiety in a dose of at least about 2 mol percent, at least about 5 mol percent, at least about 7 mol percent, or at least about 8 mol percent of the administered composition. The iodine imaging moiety can be in a dose of less than about 20 mol percent, less than about 15 mol percent, less than about 12, or less than about 10 mole percent of the administered composition.

The exact dosage will be determined by the practitioner, in light of factors related to the subject in need of diagnosis and/or treatment. Factors which may be taken into account include the severity or extent of the pulmonary condition, the general health of the subject, age, weight, and diet of the subject, as well as the timing and frequency of administration, other diagnostic and/or therapeutic techniques available and/or desirable to the subject, and/or being used by the subject, as well as reaction sensitivities, allergies, tolerance and/or response to the composition(s) of the present invention.

### EXAMPLES

A composition comprising a targeting moiety coupled to a cross-linkable moiety and coupled to an imaging moiety can be administered to a rabbit in an experimental model for imaging damaged lung tissue and/or reducing lung volume according to the present invention. In this example, the targeting moiety comprises an alpha-1 antitrypsin moiety covalently coupled to a fibrinogen moiety and covalently coupled to a Tc-99m moiety. The fibrinogen and Tc-99m moieties are each coupled to a region of the alpha-1 antitrypsin moiety other than around the Ser358 inhibitory site of alpha-1 antitrypsin, and cross-linking occurs using a thrombin moiety.

An emphysema-like condition can be induced in rabbits as follows. Under light anesthesia, rabbits can be administered porcine elastase via a nebulizer through an endotracheal tube. The treatment is repeated once weekly for four weeks. Detailed pulmonary function tests are performed under anesthesia before and after the four week treatment to determine a baseline. The rabbits can be divided into two groups, a test group and a control group.

The composition comprising a targeting moiety coupled to cross-linkable and imaging moieties can be administered to animals in the test group using an intra alveolar device (IAD). The composition is nebulized and administered to the animals via an endotracheal tube to the lungs without prior identification of areas of damaged lung tissue. The composition can be allowed about 5 to about 7 minutes to distribute and target elastase. After this time, the lungs are washed with saline and suctioned, to remove unbound compositions. The alpha-1 antitrypsin moieties target damaged lung tissue by virtue of higher amounts of elastase in areas of the lungs affected by the porcine elastase-induced condition compared with areas of the lung that are not affected or that are affected to a lesser extent. Heart rate and arterial oxygen saturation of the animals can be monitored using an oximeter and tongue probe during the procedure.

A CT scan of the lungs is then taken to image the Tc-99m moiety coupled to the alpha-1 antitrypsin moiety bound to elastase. The scan can be used to indicate and/or confirm attachment of the composition of the invention to areas of damaged lung tissue and/or the extent of damage induced.

Cross-linking is then activated by administering thrombin. Thrombin is nebulized and administered to the animal via an endotracheal tube to the lungs or to regions of the lungs identified as damaged by the CT scan. The lungs are then again suctioned using, e.g., about 120 to about 140 mmHg for about 3 to about 5 minutes, to promote collapse. Cross-linking is allowed to cure for about 7 to about 10 mins. A second CT scan of the lung can be taken to image the extent of collapse and/or sealing achieved in regions of damaged lung tissue. The lungs are then re-inflated and the animals allowed to recover from anesthesia, followed by close monitoring for about an hour.

Animals in the control group can undergo a similar procedure except that no composition of the present invention is administered. About a day after the procedure, pulmonary function tests are repeated on both the test and control groups to determine the effectiveness of lung volume reduction in the test group animals.

Pulmonary function tests include assessment of static and dynamic lung physiology as described in Ingenito et al., (2002) Bronchoscopic Lung Volume Reduction Using tissue engineering principles, American Journal of Respiratory and Critical Care Medicine, Vol. 167 pgs. 771-778 and/or Ingenito et al., “Bronchoscope volume reduction—A safe and effective alternative to surgical therapy for emphysema,” American Journal of Respiratory and Critical Care Medicine, Vol 164 pgs 295-301 (2001).

Little change is expected in control group animals in QSPV profiles. Animas in the test group, however, are expected to show significant reductions in RV and TLC coupled with a significant increase in RV/TLC ratios. A significant decrease in vital capacity and airway resistance is also expected in test group animals as compared with controls. Further, few post-procedural complications are expected due to the minimal invasiveness of the procedure. For example, low incidence and severity of fevers, respiratory distress, wound infections and/or death would be expected.

While preferred embodiments of the present invention have been shown and described herein, it will be obvious to those skilled in the art that such embodiments are provided by way of example only. Numerous variations, changes, and substitutions will now occur to those skilled in the art without departing from the invention, and it should be understood that various alternatives to the embodiments of the invention described herein may be employed in practicing the invention. It is intended that the following claims define the scope of the invention and that methods and compositions within the scope of these claims, along with their equivalents, are covered thereby.

All publications, patents, and patent applications mentioned in this specification are herein incorporated by reference to the same extent as if each individual publication, patent or patent application was specifically and individually indicated as being incorporated by reference.

1. A method of imaging damaged lung tissue, comprising:
administering to a subject in need thereof a composition comprising an imaging moiety and a targeting moiety wherein said moieties are coupled and wherein said targeting moiety targets damaged lung tissue; and
imaging said damaged lung tissue.

2. The method as recited in claim 1 wherein said lung tissue comprises epithelial lining fluid.

3. The method as recited in claim 1 wherein said composition does not comprise a polysaccharide or a carbohydrate moiety.

4. The method as recited in claim 1 wherein said composition does not comprise an antibody.

5. The method as recited in claim 1 wherein said composition does not comprise a mutant plasminogen activator-inhibitor type 1.

6. The method as recited in claim 1 wherein said composition does not comprise a lung membrane dipeptidase-binding molecule.

7. The method as recited in claim 1 wherein said targeting moiety does not target a lung membrane dipeptidase.

8. The method as recited in claim 1 wherein said targeting moiety targets a damage-correlated moiety.

9. The method as recited in claim 8 wherein said damage-correlated moiety comprises a cell surface marker.

10. The method as recited in claim 8 wherein said damage-correlated moiety comprises an ECM component.

11. The method as recited in claim 1 wherein said targeting moiety targets elastase.

12. The method as recited in claim 1 wherein said targeting moiety targets neutrophil elastase.

13. The method as recited in claim 1 wherein said targeting moiety comprises a protease inhibitor moiety.

14. The method as recited in claim 1 wherein said targeting moiety comprises an alpha-1 antitrypsin moiety.

15. The method as recited in claim 14 wherein said alpha-1 antitrypsin moiety is a recombinant alpha-1 antitrypsin moiety.

16. The method as recited in claim 1 wherein said targeting moiety comprises an elafin moiety.

17. The method as recited in claim 16 wherein elafin moiety is a recombinant elafin moiety.

18. The method as recited in claim 1 wherein said targeting moiety comprises a serpin moiety.

19. The method as recited in claim 18 wherein said serpin moiety is a recombinant serpin moiety.

20. The method as recited in claim 18 wherein said serpin moiety is a secretory leukoprotease inhibitor (SLP1) moiety.

21. The method as recited in claim 20 wherein said secretory leukoprotease inhibitor (SLP1) moiety is a recombinant secretory leukoprotease inhibitor (SLP1) moiety.

22. The method as recited in claim 1 wherein said targeting moiety targets at least one matrix metalloproteinase selected from MMP-1, MMP-2, MMP-3, MMP-4, MMP-5, MMP-6, MMP-7, MMP-8, and MMP-9.

23. The method as recited in claim 22 wherein said composition does not comprise a hyaluronic acid or a salt thereof.

24. The method as recited in claim 1 wherein said targeting moiety targets desmosine and/or isodesmosine.

25. The method as recited in claim 1 wherein said targeting moiety targets CD8 and/or CD4.

26. The method as recited in claim 1 wherein said targeting moiety targets a smoke-related moiety.

27. The method as recited in claim 1 wherein said imaging moiety comprises a contrasting agent.

28. The method as recited in claim 27 wherein said contrasting agent is non-ionic.

29. The method as recited in claim 27 wherein said contrasting agent is ionic.

30. The method as recited in claim 1 wherein said imaging moiety comprises at least one metal compound selected from a tantalum compound and a barium compound.

31. The method as recited in claim 1 wherein said imaging moiety comprises iodine.

32. The method as recited in claim 1 wherein said imaging moiety comprises at least one organic iodo acid selected from an iodo carboxylic acid, a triiodophenol, an iodoform, and a tetraiodoethylene.

33. The method as recited in claim 1 wherein said imaging moiety comprises a non-radioactive moiety.

34. The method as recited in claim 1 wherein said imaging moiety comprises a proton-emitting moiety.

35. The method as recited in claim 1 wherein said imaging moiety comprises a radiopaque moiety and/or a radioactive moiety.

36. The method as recited in claim 1 wherein said imaging moiety comprises a magnetic moiety.

37. The method as recited in claim 1 wherein said imaging moiety comprises a radiopharmaceutical.

38. The method as recited in claim 1 wherein said imaging moiety comprises an In-111 moiety.

39. The method as recited in claim 1 wherein said imaging moiety comprises a Tc-99m moiety.

40. The method as recited in claim 1 wherein said imaging moiety comprises an Xe-133 moiety.

41. The method as recited in claim 1 wherein said composition is less than 10 microns.

42. The method as recited in claim 1 wherein said composition is less than 5 microns.

43. The method as recited in claim 1 wherein said composition is less than 1 micron.

44. The method as recited in claim 1 wherein said administering is carried out via inhalation.

45. The method as recited in claim 44 wherein said inhalation is carried out via the mouth.

46. The method as recited in claim 1 wherein said administering is carried out via trans-thoracic administration.

47. The method as recited in claim 1 wherein said administering is carried out via intravenous administration.

48. The method as recited in claim 1 wherein said imaging of said damaged lung tissue is carried out via a radiological technique.

49. The method as recited in claim 48 wherein said radiological technique is at least one selected from an X-ray, a CT scan, a PET scan, a nuclear scan, a SPECT, and a scintigraphy.

50. The method as recited in claim 1 wherein said composition further comprises a coupled cross-linkable moiety and/or another coupled targeting moiety.

51. The method as recited in claim 50, further comprising cross-linking and/or sealing said damaged lung tissue.

52. The method as recited in claim 1, further comprising administering a washing moiety.

53. A method of diagnosing a pulmonary condition comprising:
administering to a subject a composition comprising an imaging moiety and a targeting moiety wherein said moieties are coupled and wherein said targeting moiety targets damaged lung tissue; and
imaging said damaged lung tissue.

54. The method as recited in claim 53 wherein said lung tissue comprises epithelial lining fluid.

55. The method as recited in claim 53 wherein said pulmonary condition is emphysema.

56. The method as recited in claim 53 wherein said pulmonary condition is COPD.
